SEQUENCE LISTING (6 sequences)

SEQ ID NO: 1 - DNA, 24 bp, Artificial Sequence
  primer
atgtaatgatgagcactctaacgg

SEQ ID NO: 2 - DNA, 20 bp, Artificial Sequence
  primer
ggcttttgagattggcatcc

SEQ ID NO: 3 - DNA, 21 bp, Artificial Sequence
  primer
gagagctgcagggccctttgc

SEQ ID NO: 4 - DNA, 25 bp, Artificial Sequence
  primer
ctccctggtttctcttcccaagacc

SEQ ID NO: 5 - DNA, 19 bp, Artificial Sequence
  primer
gtgccagcmgccgcggtaa

SEQ ID NO: 6 - DNA, 20 bp, Artificial Sequence
  primer
ccgycaattymtttragttt

(19) United States
(12) Patent Application Publication
(10) Pub. No.: US 2023/0087012 A1
(43) Pub. Date: Mar. 23, 2023

(54) TRANSFERABLE MICROBIOTA FOR THE TREATMENT OF ULCERATIVE COLITIS

(71) Applicant: Cornell University, Ithaca, NY (US)

(72) Inventors: Randy LONGMAN, New Rochelle, NY (US); Lasha GOGOKHIA, Ithaca, NY (US); Svetlana LIMA, Ithaca, NY (US)

(21) Appl. No.: 17/904,074

(22) PCT Filed: Feb. 15, 2021
(86) PCT No.: PCT/US21/18131

§ 371 Date: Aug. 11, 2022

Related U.S. Application Data
(60) Provisional application No. 62/976,739, filed on Feb. 14, 2020.

(51) Int. Cl. A61K 35/741; C12N 1/20; A61P 1/04; A61K 35/744; A61K 35/745; A61K 35/742

(52) U.S. Cl. CPC A61K 35/741; C12N 1/20; A61P 1/04; A61K 35/744; A61K 35/745; A61K 35/742; A61K 2035/115

(57) ABSTRACT

Provided are compositions and methods for treatment of inflammatory bowel disease. The compositions comprise Odoribacter and/or Alistipes bacteria, identified herein as being core transferable bacteria in fecal matter transfer therapies. Methods are also provided comprising administering to an individual suffering from IBD a composition comprising Odoribacter and/or Alistipes bacteria.

### CROSS-REFERENCE TO RELATED APPLICATIONS

This application claims priority to U.S. Provisional patent application No. 62/976,739, filed on Feb. 14, 2020, the disclosure of which is incorporated herein by reference.

### BACKGROUND OF THE DISCLOSURE

It is estimated that over two million people worldwide suffer from ulcerative colitis (UC). Biologic therapy has significantly improved treatment for ulcerative colitis, but nearly two-thirds of patients attenuate response. Additional therapeutic modalities are therefore needed to address the underlying pathophysiology of UC. Fecal microbiota transplant (FMT) is an emerging therapy for the treatment of UC, but several randomized controlled trials have shown variable efficacy of FMT, and because the microbial mechanisms responsible for clinical response are not well understood, rational design or selection of specific microbial strains or communities has heretofore not been possible. A lack of mechanistic understanding of microbial transferability, engraftment and immune cell impact underlying the efficacy of FMT is a clear limitation to the use of FMT.

### SUMMARY OF THE DISCLOSURE

In this disclosure, identification of transferable microbiota and IgA-coated microbiota in recipients of fecal transplants for ulcerative colitis (UC) that had clinical improvement was carried out. Based at least in part on these observations, the disclosure provides compositions for treatment of inflammatory bowel diseases, such as UC. The compositions comprise one or more types of bacteria that are identified in this disclosure as core transferable microbiota. For example, the composition comprises, consists essentially of, or consists of Odoribacter and/or Alistipes bacteria. In embodiments, the only bacteria in the composition may be Odoribacter and/or Alistipes bacteria. In embodiments the Odoribacter bacteria are Odoribacter splanchnicus, and the Alistipes bacteria are Alistipes shahii. The compositions may further comprise a fiber component. The compositions may be in any form, solid, liquid, suspension, gel, paste, freeze dried, powdered or any combinations or variations thereof. In embodiments, pharmaceutical compositions comprising or consisting essentially of Odoribacter and/or Alistipes bacteria are provided. In embodiments, the pharmaceutical composition comprises, or consists essentially of Odoribacter splanchnicus and/or Alistipes shahii and optionally further comprises one or more of Streptococcus salivarius, Ruminococcus bromii, Roseburia inulinivorans, Roseburia intestinalis, Roseburia hominis, Lachnospiraceae bacterium 5 1 63FAA, Eubacterium ramulus, Eubacterium eligens, Eubacterium biforme, Coprococcus catus, Collinsella aerofaciens, Catenibacterium mitsuokai, Bilophila unclassified, Bifidobacterium longum, Bifidobacterium adolescentis, Barnesiella intestinihominis, Bacteroides massiliensis, and Bacteroides dorei.

The disclosure also provides a method for treatment of an inflammatory bowel disease, such as UC, comprising administering to an individual in need of treatment a composition comprising the core transferable bacteria described herein. For example, the method may comprise administering an in individual in need of treatment one or more of Streptococcus salivarius, Ruminococcus bromii, Roseburia inulinivorans, Roseburia intestinalis, Roseburia hominis, Lachnospiraceae bacterium 51 63FAA, Eubacterium ramulus, Eubacterium eligens, Eubacterium biforme, Coprococcus catus, Collinsella aerofaciens, Catenibacterium mitsuokai, Bilophila unclassified, Bifidobacterium longum, Bifidobacterium adolescentis, Barnesiella intestinihominis, Bacteroides massiliensis, and Bacteroides dorei. In embodiment, the method comprises administering to an individual in need of treatment a composition comprising Odoribacter and/or Alistipes bacteria, where these may be the only bacteria present in the composition. The bacteria or the compositions comprising the bacteria may be administered orally or as an enema or as an infusion into the upper gastrointestinal tract. The amount of bacteria delivered per dose may be from 10 million to 100 billion.

### BRIEF DESCRIPTION OF THE DRAWINGS

FIG. 1. Clinical response to FMT is associated with a core set of transferable microbiota. (A) Principal coordinate analysis plot is shown using Bray-Curtis and stratified by donor (DON), recipient pre (PRE) and at week 4 (WK4) post FMT. P-value is shown, Monte Carlo, PERMANOVA. (B) Venn diagram depicting the number of bacterial species unique to a single variable (DON, PRE or WK4) or shared by two, or all three variables. Microbial cut off for prevalence was defined as ≥50% and relative abundance as ≥0.1%. In total 342 species did not enter any of the core groups shown in the Venn diagram. (C) Heatmap depicting the relative abundance of the 17 species defined by the Venn diagram as being part of the core transferable microbes (CTM: core shared microbes between DON and WK4 post FMT). (D) Boxplot comparing the median relative abundance of the CTM in DON, PRE and WK4 post FMT. P values indicated, Wilcoxon-paired test followed by Bonferroni multiple comparison correction test. (E) Frequency of bacterial strains belonging to the 17 acquired microbes shown in figure B and C. Strains were categorized as unique to the donor, unique to the patient, shared by the donor and patient, and undetected strains (absent in PRE and DON, but present in WK4). Strain classification was generated by Strain Finder Python package.(F) Boxplot comparing median relative abundance of the CTM in responders or non-responders. P-value, Mann-Whitney non-parametric test. (G) Responders Venn diagram depicting the number of bacterial species unique to a single variable (Responders: DON, PRE or WK4) or shared by two, or all three variables. Microbial cut off for prevalence was defined as ≥50% and relative abundance as ≥0.1%. In total 236 species did not enter any of the core groups shown in the responders Venn diagram. (H) Heatmap depicting the relative abundance of the 20 species defined as being part of the core shared microbes between responders DON and WK4 post FMT. (I) Boxplot comparing the median relative abundance of the responders' CTM in responders or non-responders: DON, PRE and WK4 post FMT. P values indicated, Wilcoxon-paired test followed by Bonferroni multiple comparison correction test. (J) Correlation between A CTM unique to responders and UC clinical improvement index (Δ Mayo). Linear regression analyses reveal a significant correlation for Δ CTM unique to responders and A Mayo (P=0.0014). (K) Mean frequency of strains belonging to CTM unique to responders and shown in figure H in bold. Strains are categorized as being unique to the donor, unique to the patient, shared by the donor and patient, and undetected strains (absent in PRE and DON, but present in WK4). Strain classification was generated by Strain Finder Python package.

FIG. 2. IgA-seq identifies donor-derived Odoribacter as immune reactive microbe associated with clinical response to FMT. (A and B) Human cohort IgA-sequencing. (C and D) Humanized mouse model (HMM). (A) IgA-sequencing and microbial isolation strategy are shown. Fecal homogenate was filtered, blocked, and stained with cell-permeable DNA dye Syto BC and isotype or anti-IgA antibody. Samples were gated on the basis of forward scatter (FSC) and side scatter (SSC). (B) Heatmap depicting relative abundance of the 29 IgA coated bacteria identified as being part of TIM (core shared microbes between DON and WK4 post FMT). (C) Humanized mouse model (HMM) experimental design and IgA-sequencing strategy are shown. Humanized mice (HM) received fecal transplant from the 2-donor FMP of the human cohort. Fecal homogenate was filtered, blocked, and stained with cell-permeable DNA dye Syto BC and anti-IgA antibody. Samples were gated on the basis of forward scatter (FSC) and side scatter (SSC). (D) Boxplots display the six bacterial genera significantly higher abundant in IgA+ samples when compared to IgA− samples. Mann-Whitney, FDR− adjusted P-value<0.1. (E) Correlation between TIM found to be enriched in the mouse IgA coated community and UC clinical improvement index (Δ Mayo). Linear regression analyses reveal a significant correlation for A relative abundance of Odoribacter genus and UC clinical improvement index (Δ Mayo, P=0.005).

FIG. 3. Patient-derived O. splanchinicus induces iTregs and alleviate T cell colitis. (A-D) Germ-free C57BL/6 mice were colonized with 2×10⁹ CFU of patient-derived O. splanchnicus and analyzed after 20 days. (A and B) Flow cytometry of live, CD4+ T cells was used to evaluate RORgt and Foxp3 expression in large intestine lamina propria. (B) Mean percentages are shown for each experimental group, and error bars reflect SEM. P values indicated, T-test. Data are compiled from three independent experiments. (C and D)

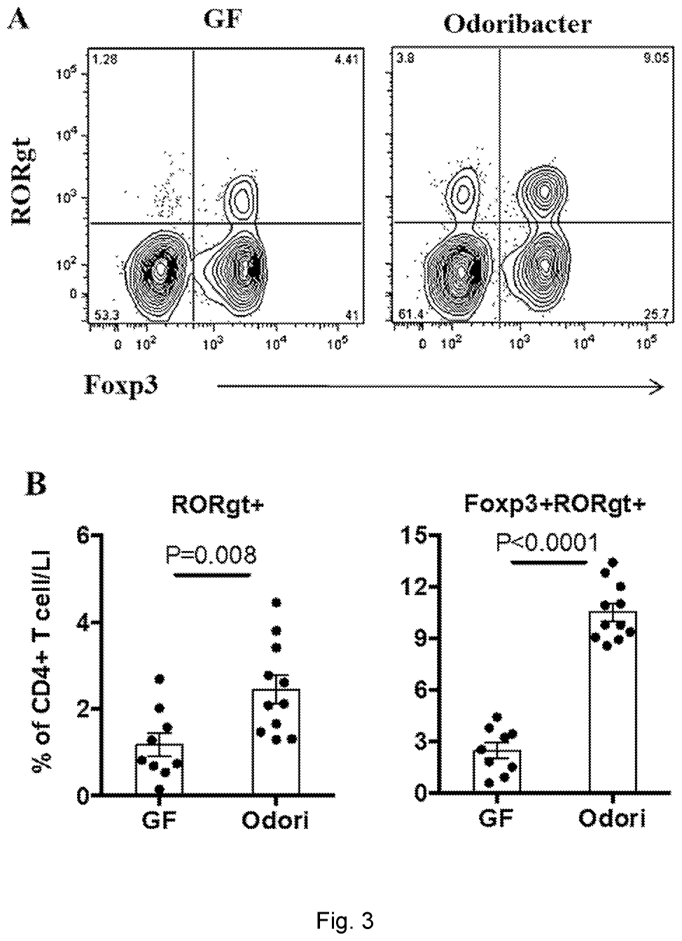

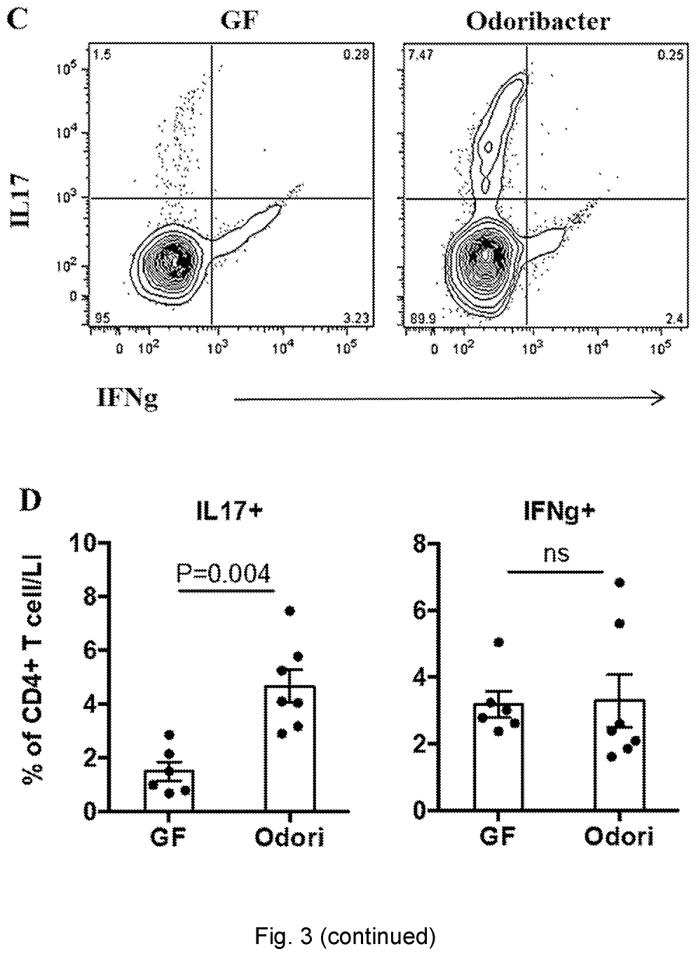

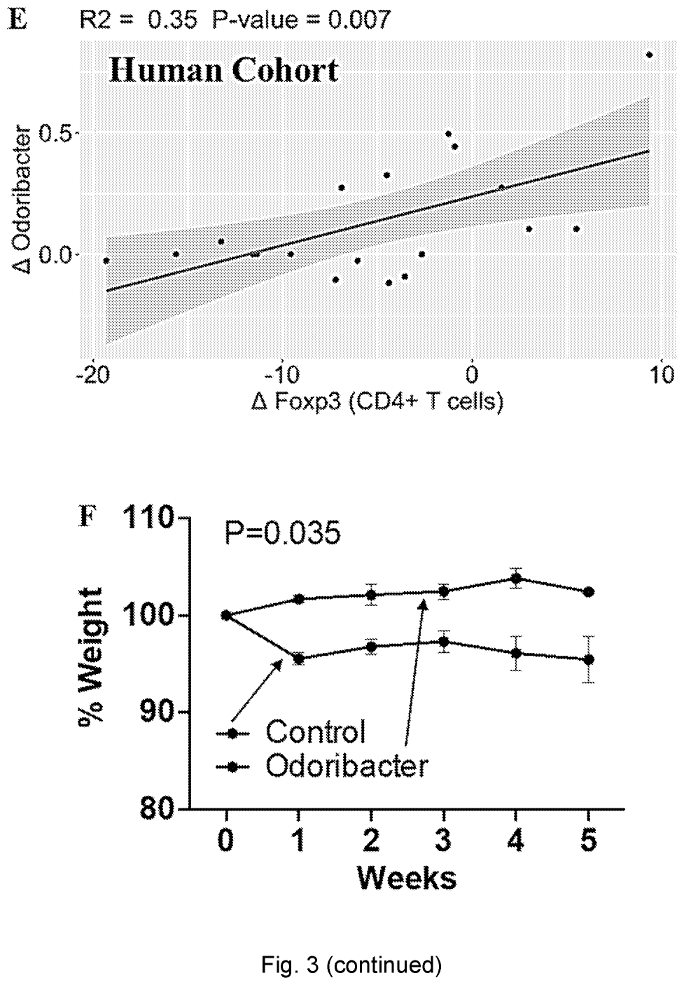

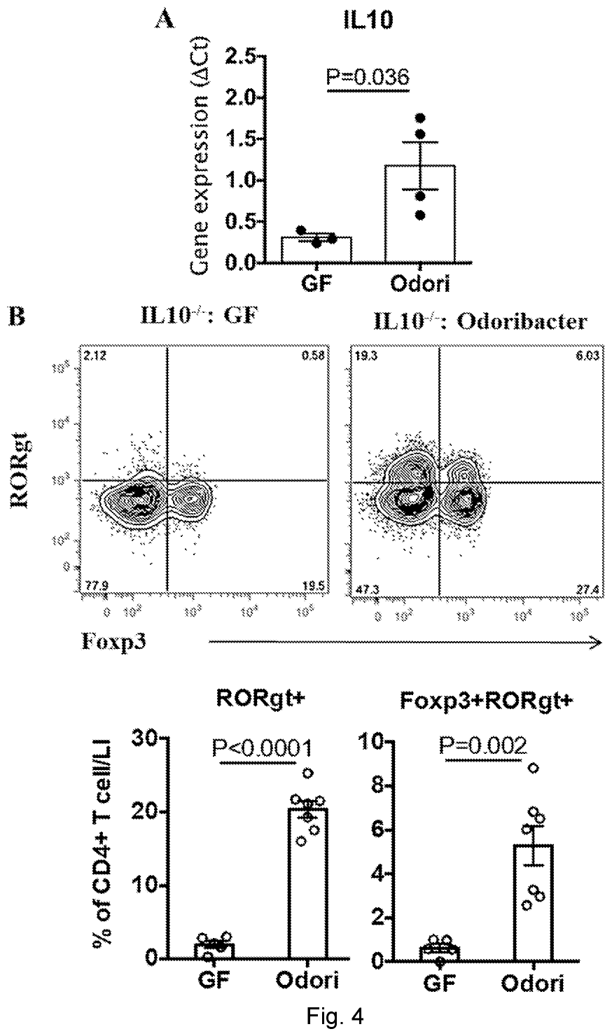

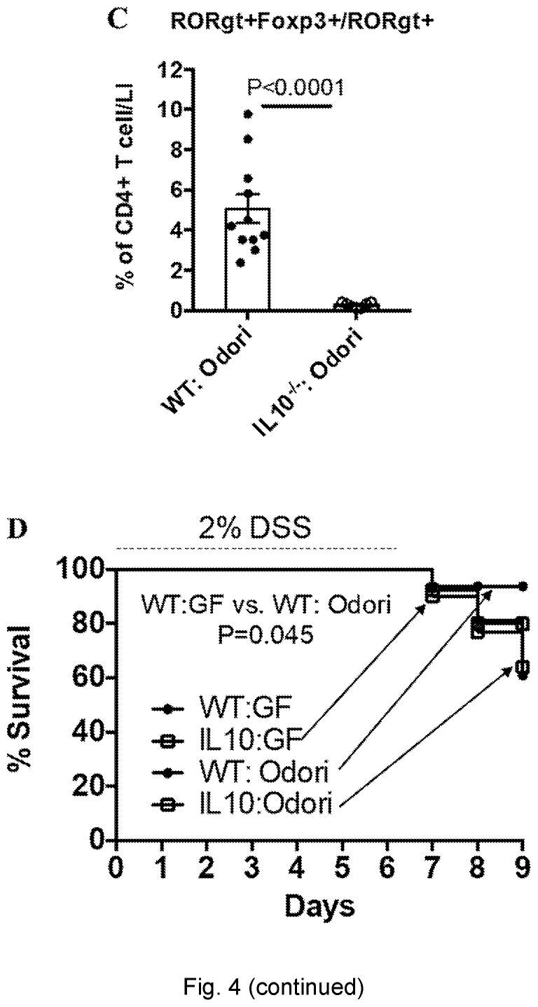

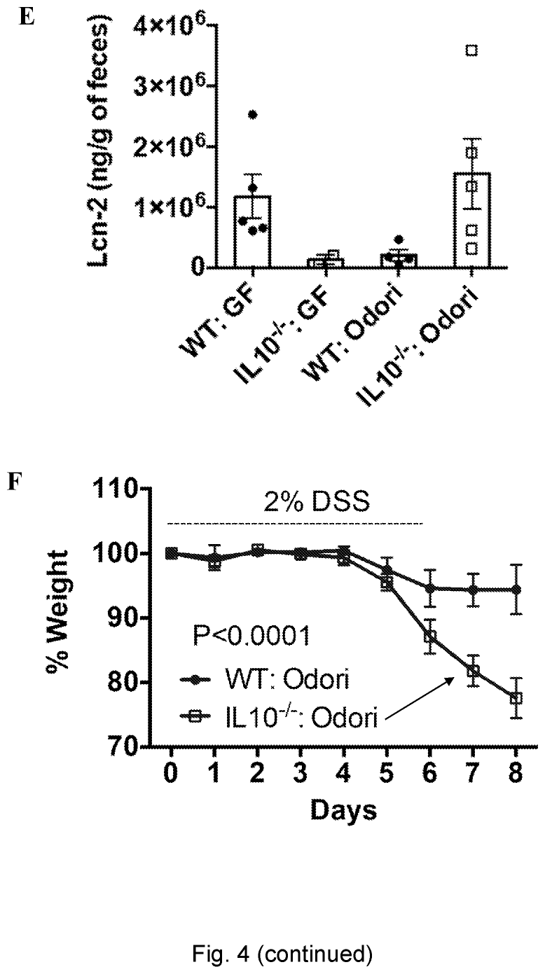

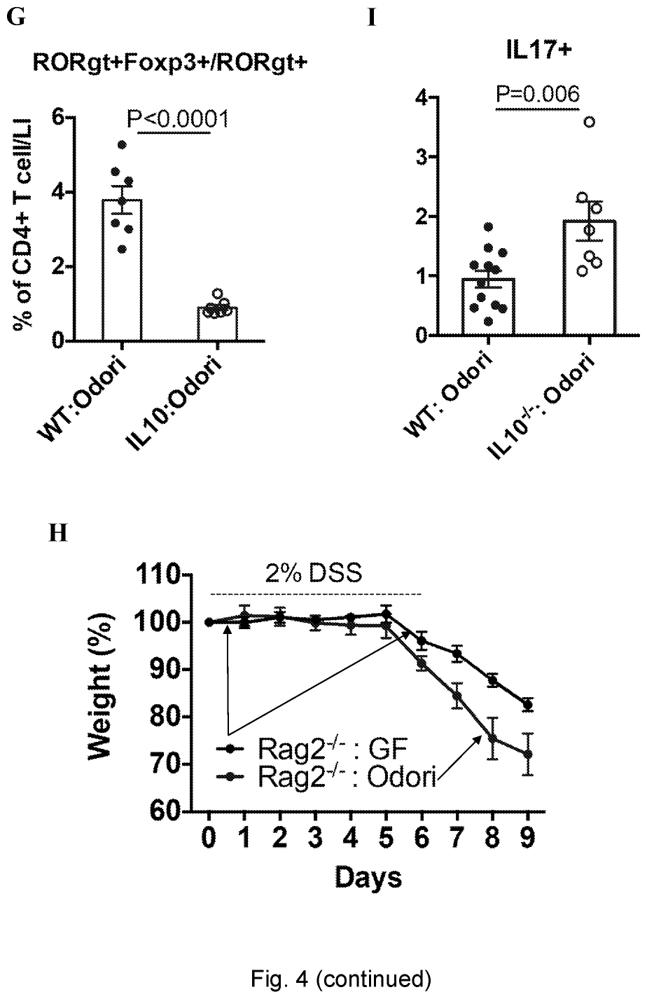

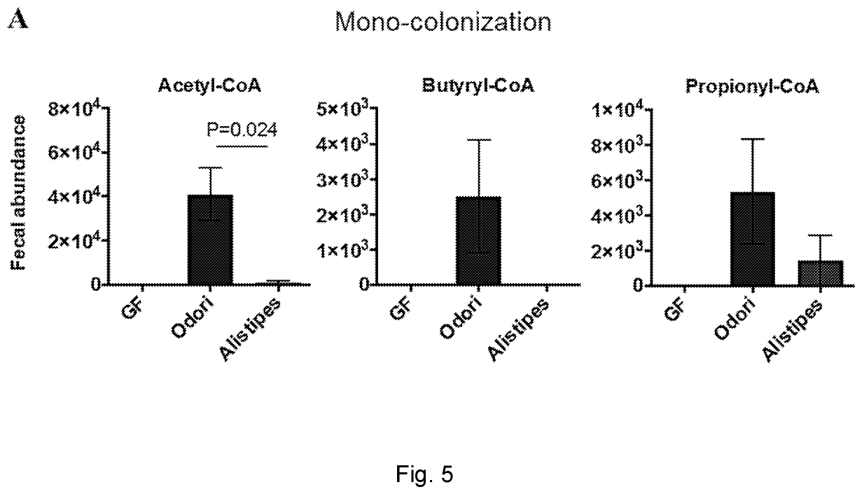

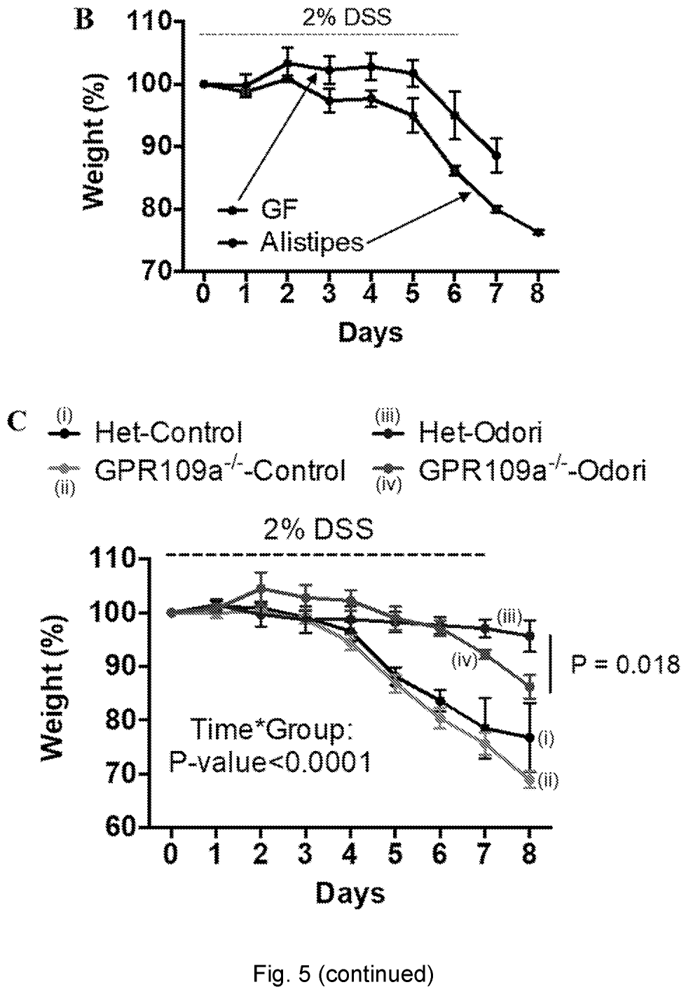

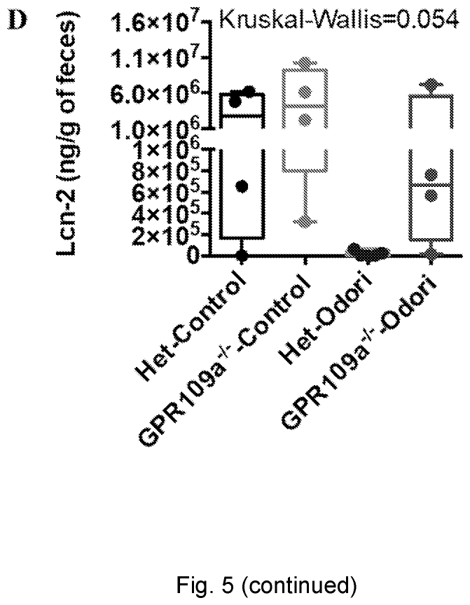

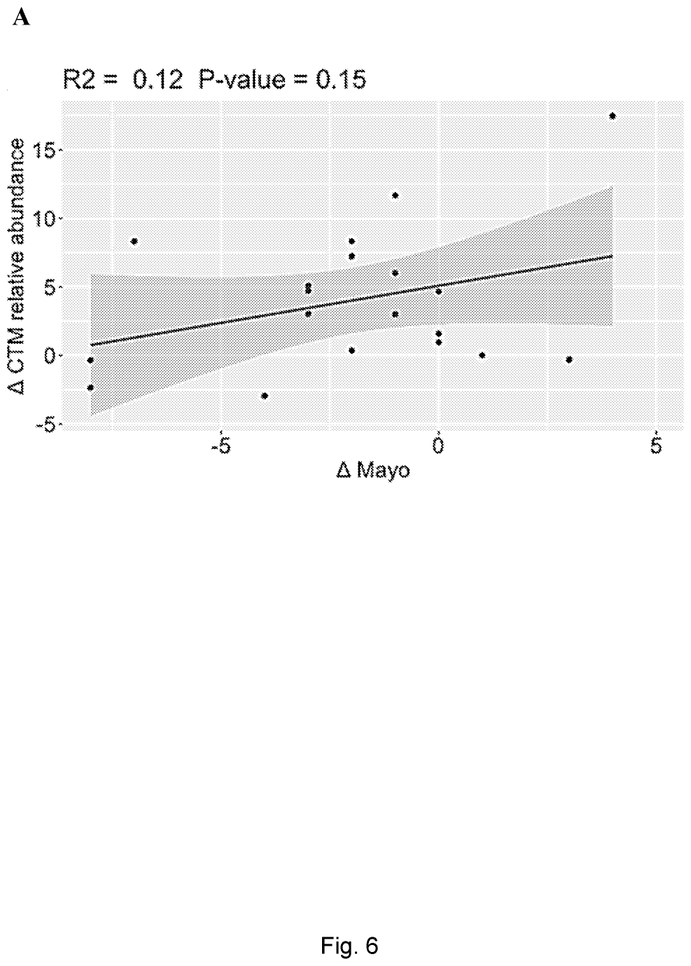

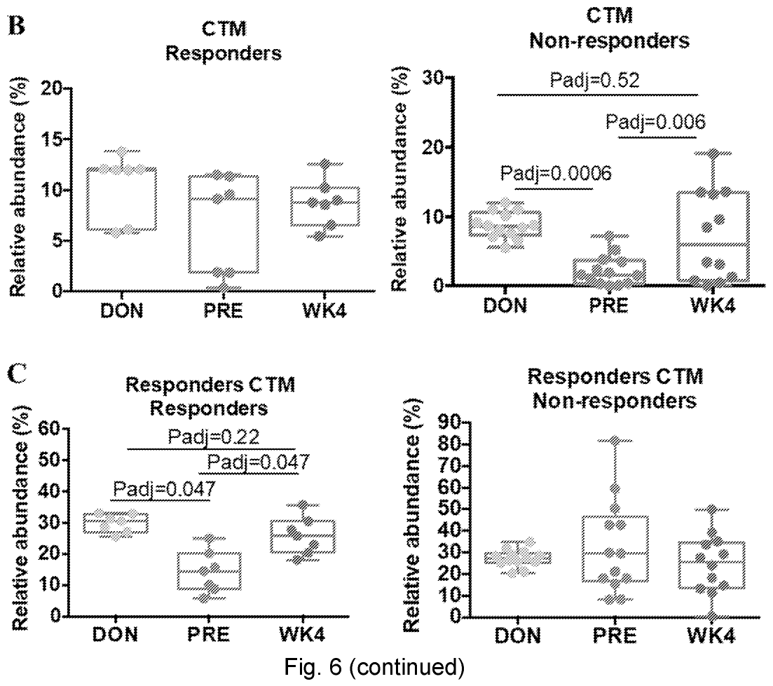

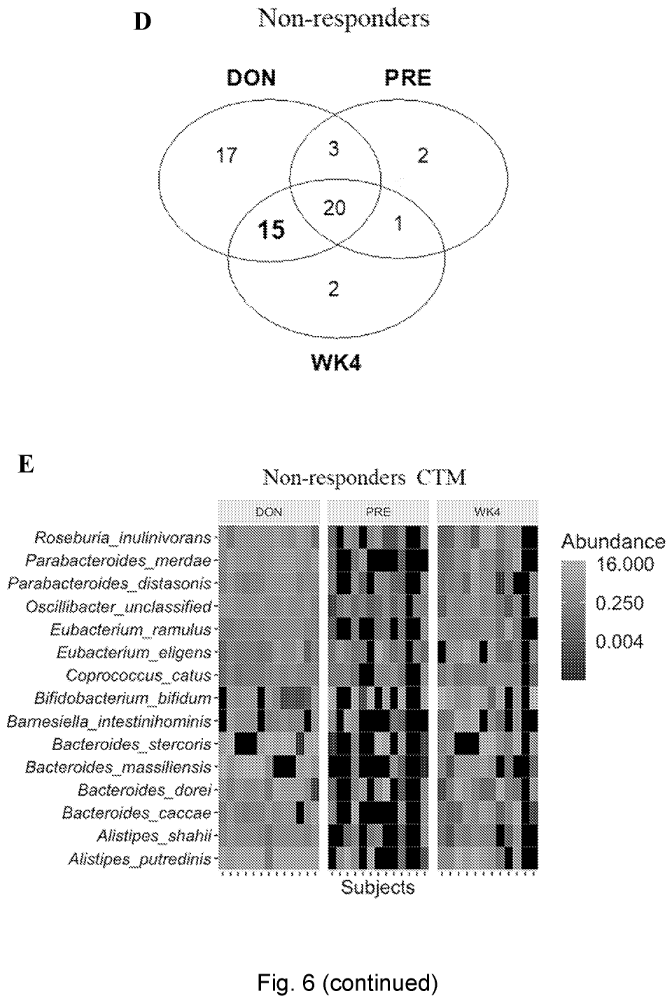

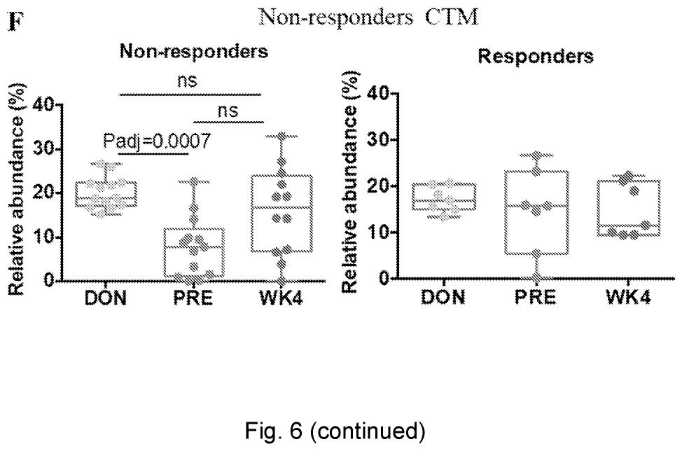

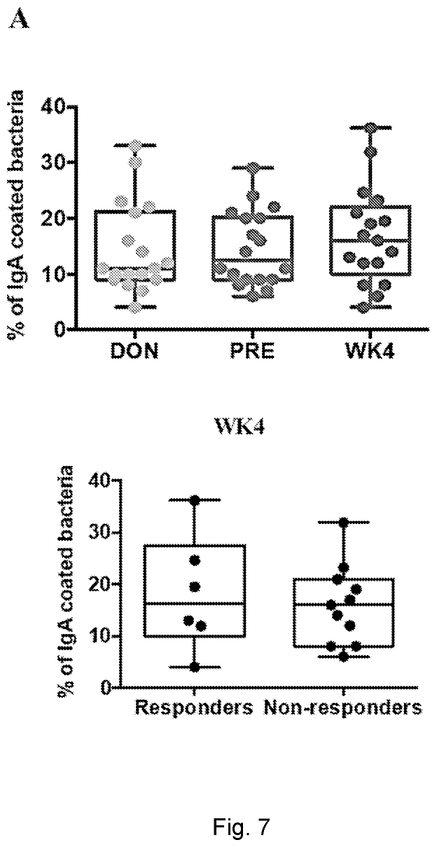

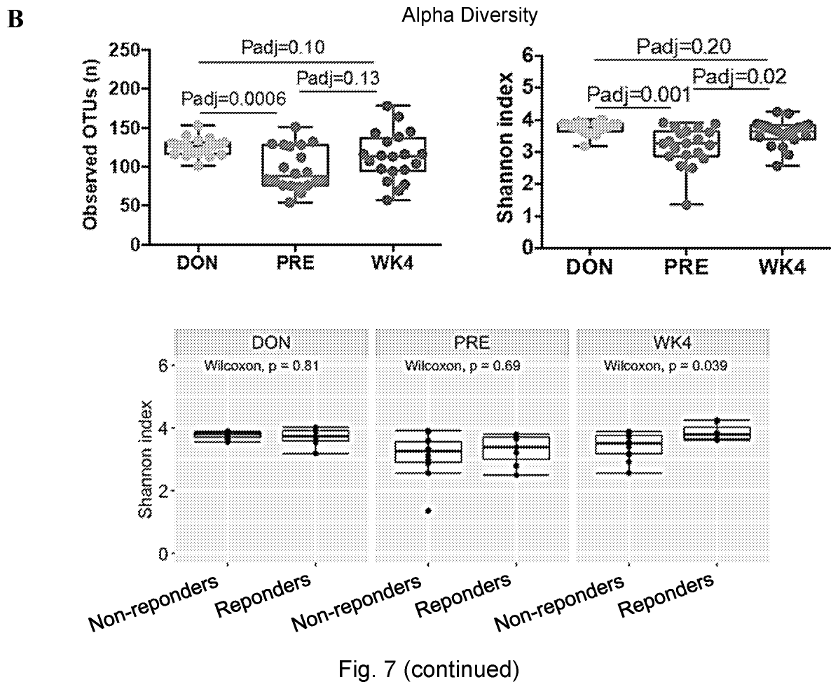

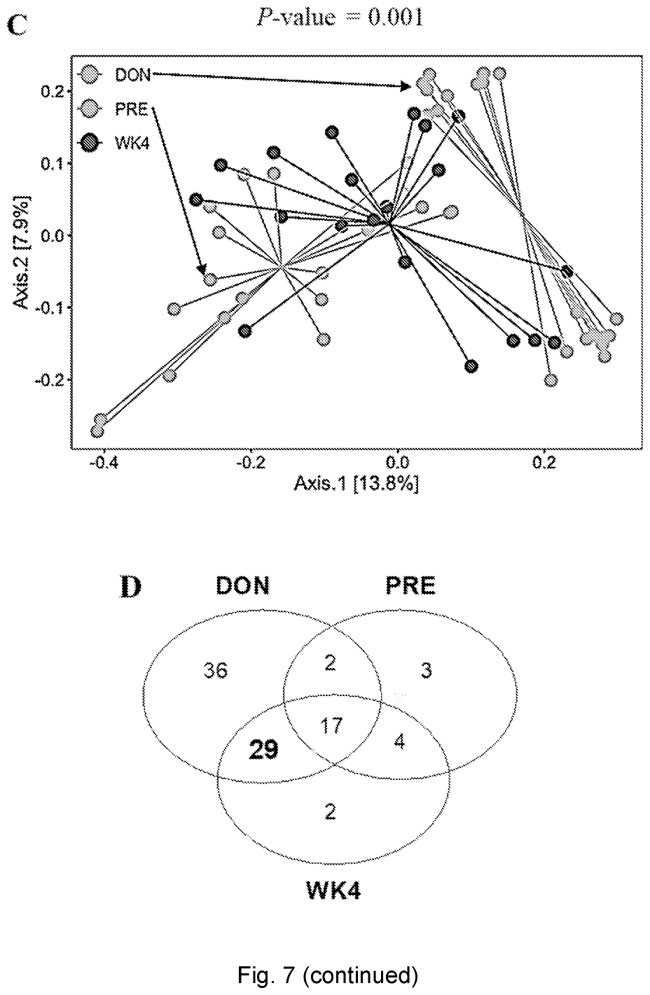

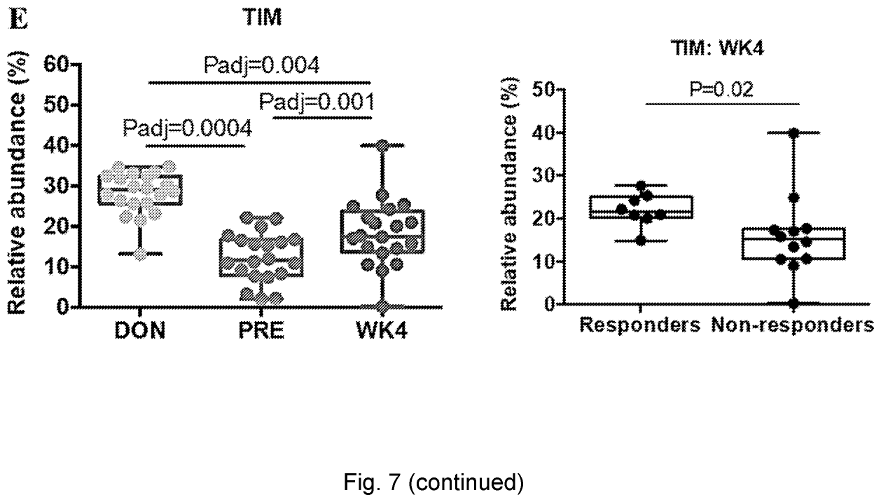

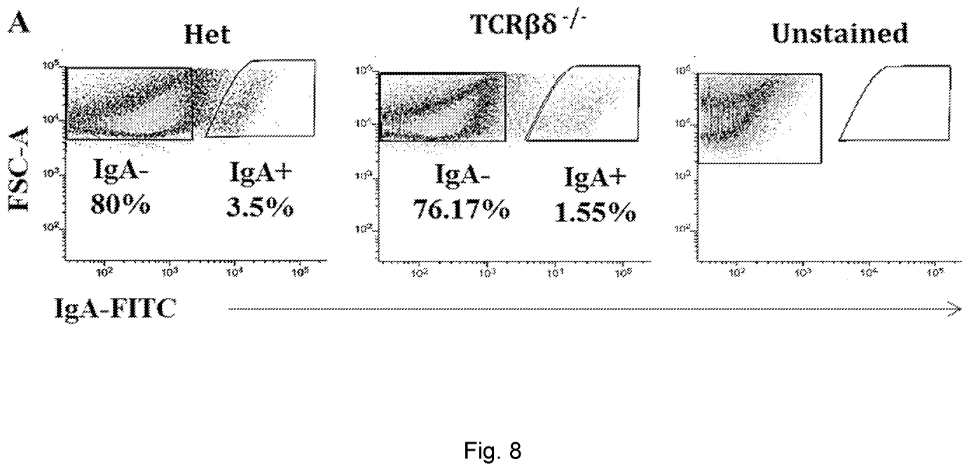

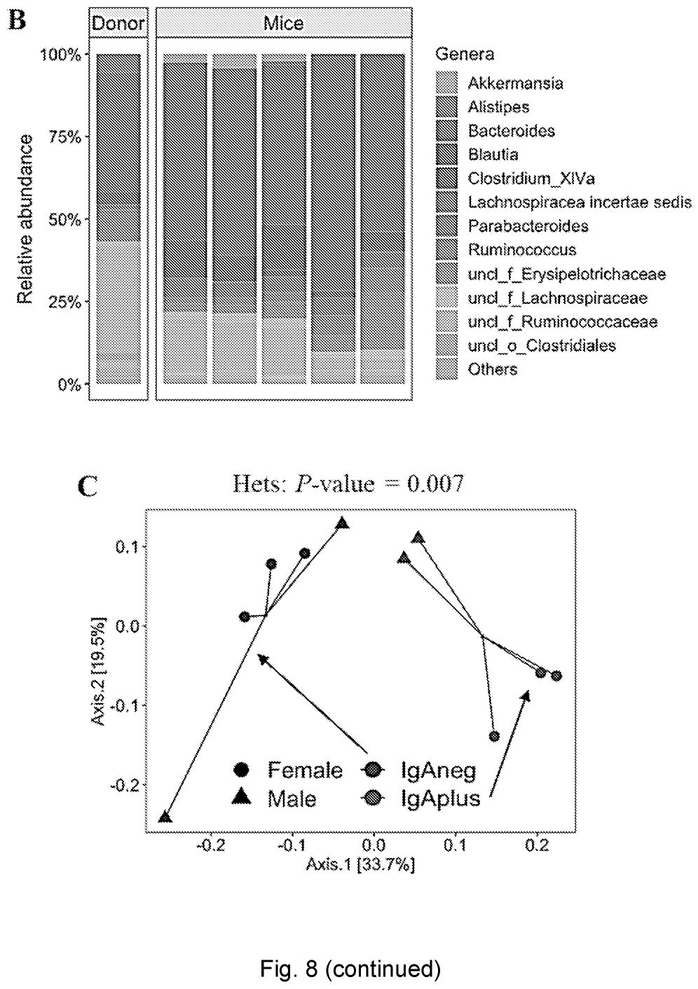

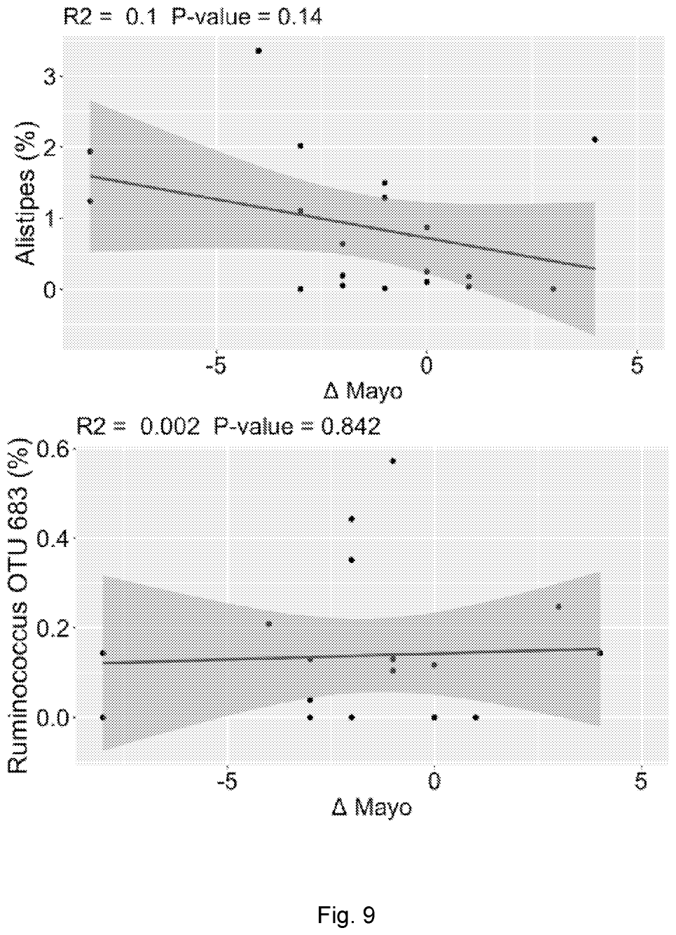

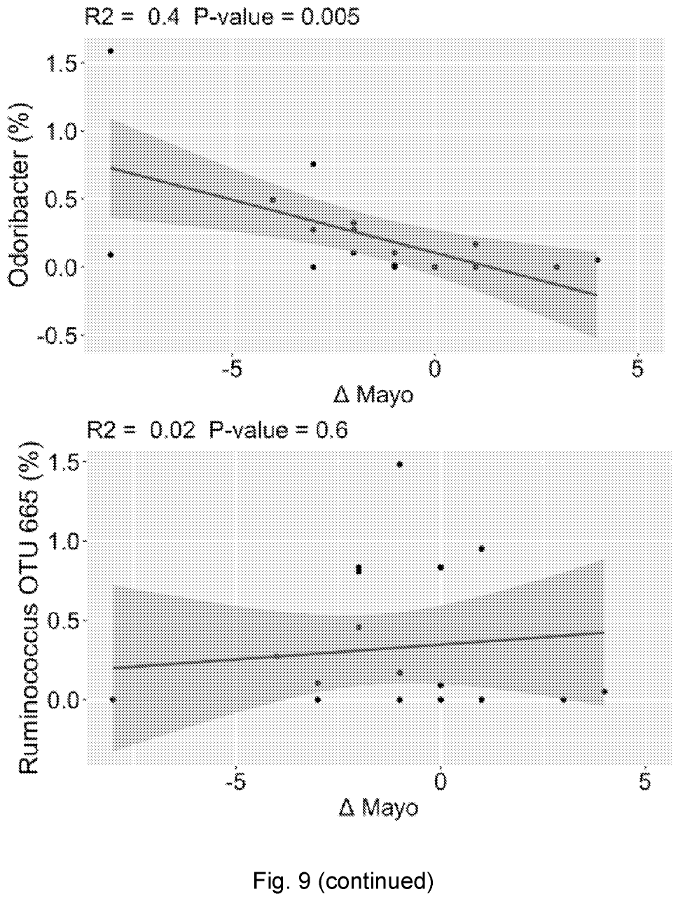

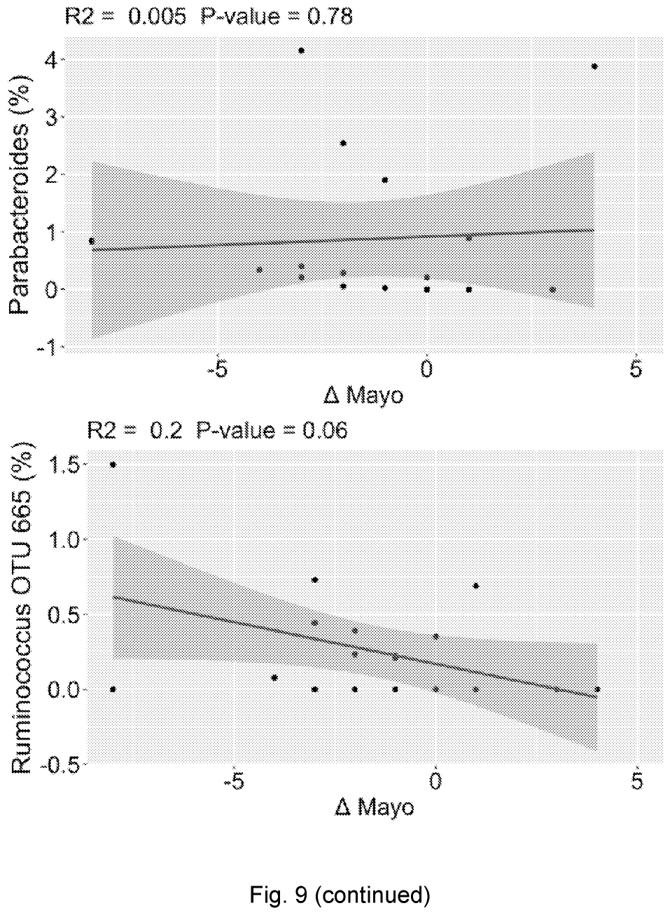

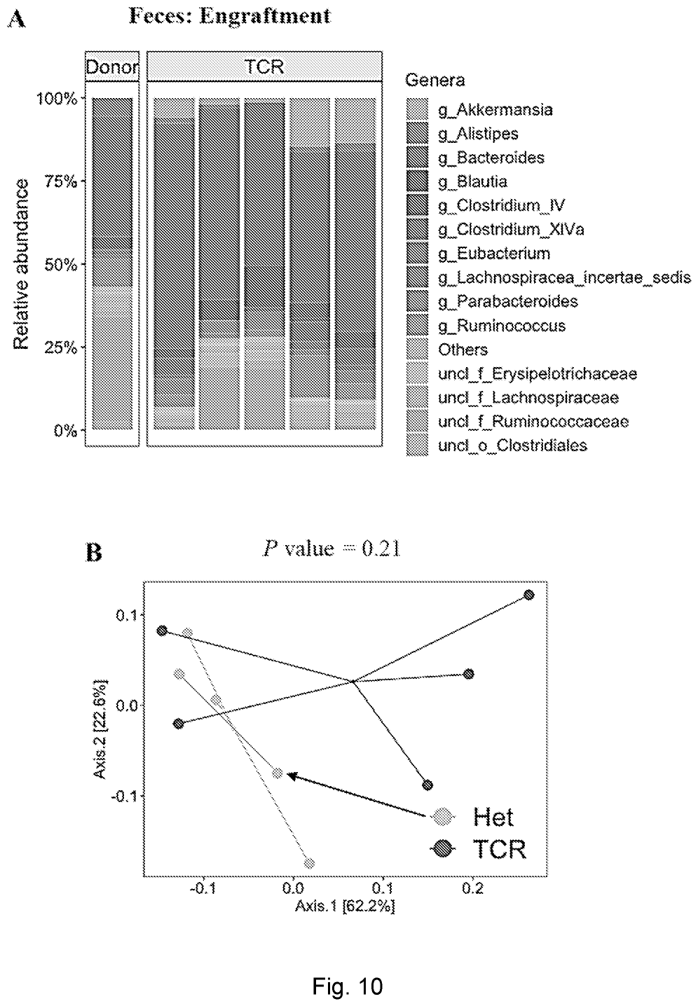

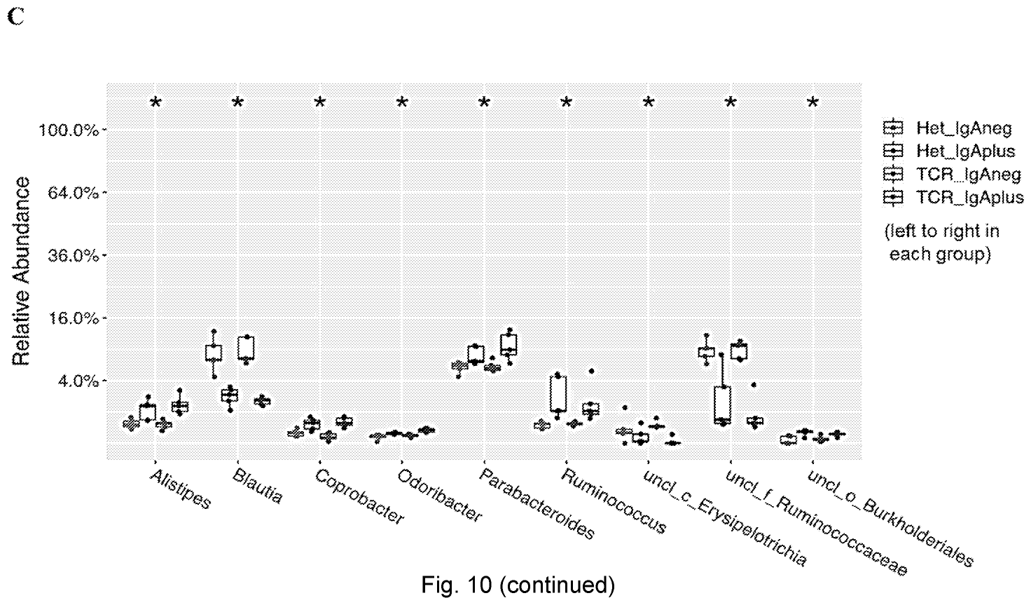

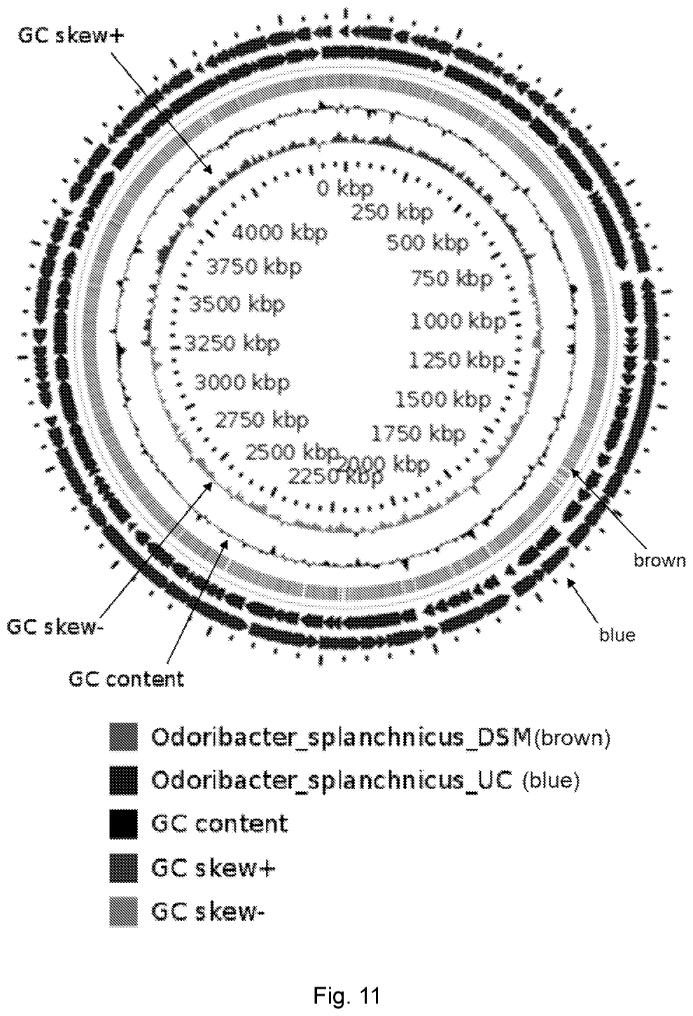

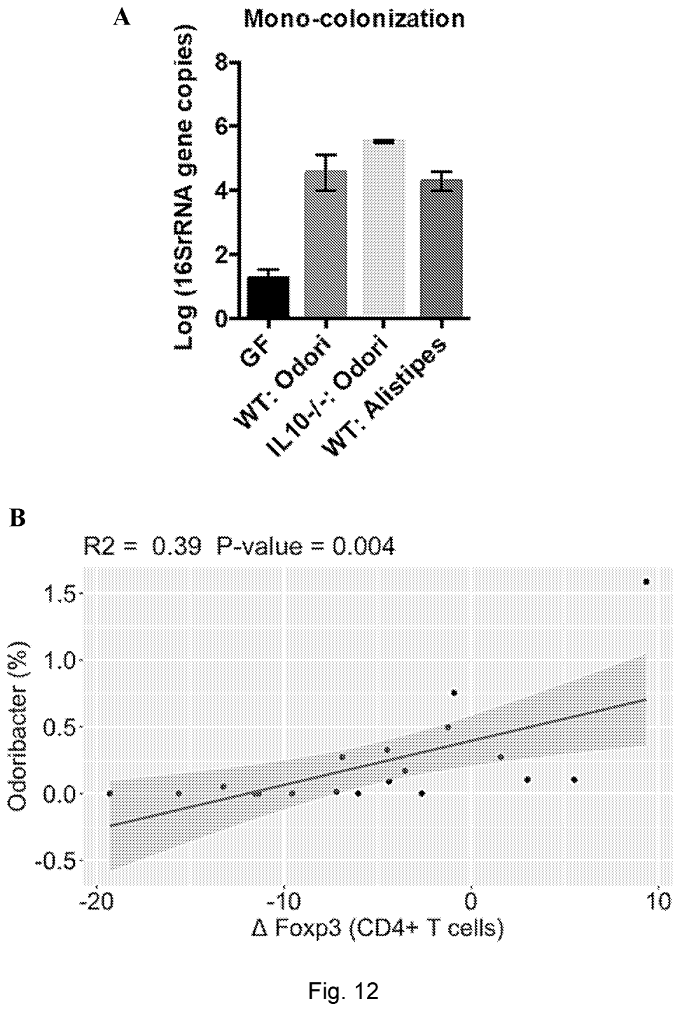

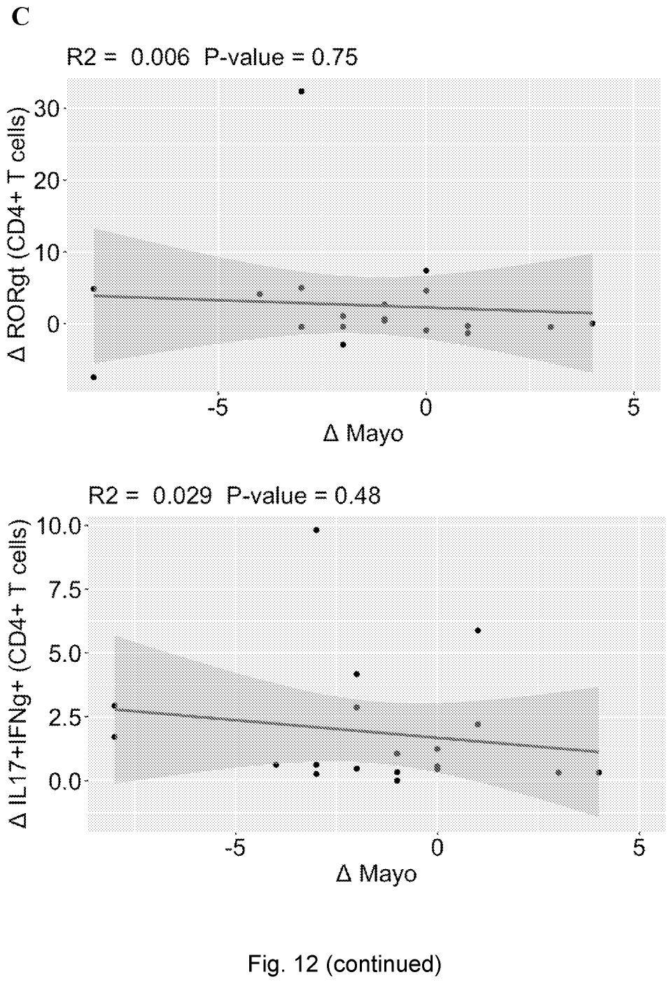

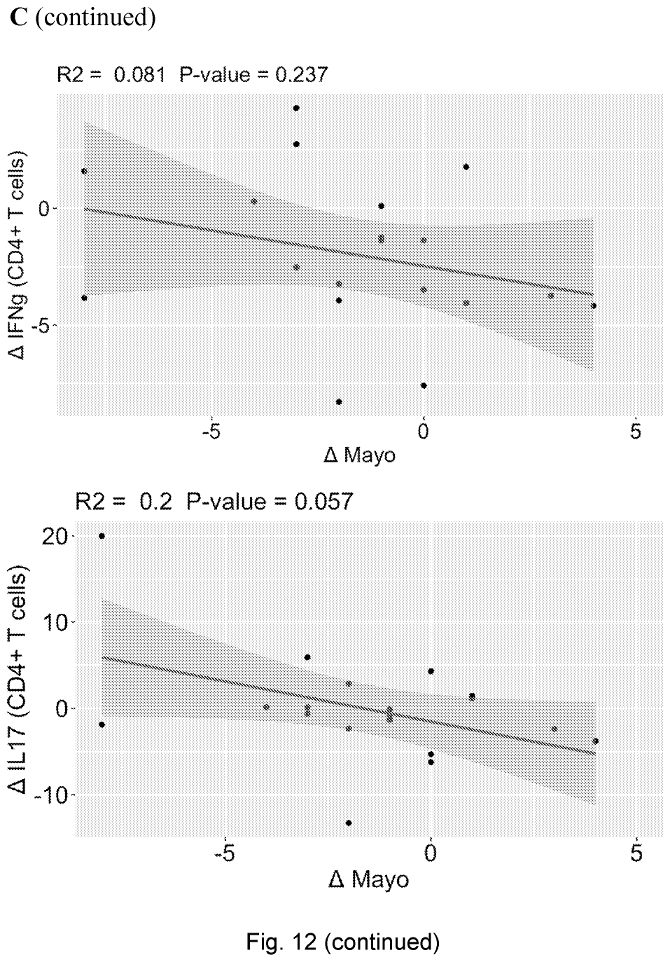

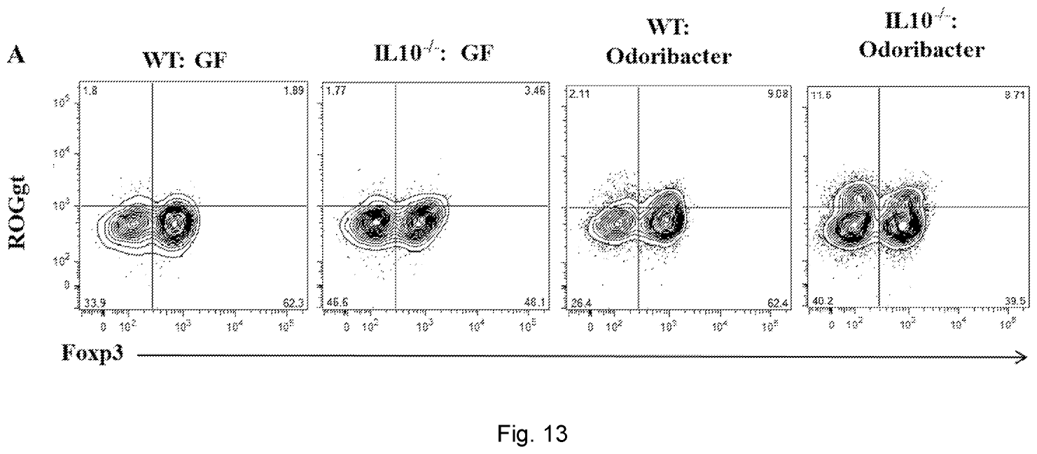

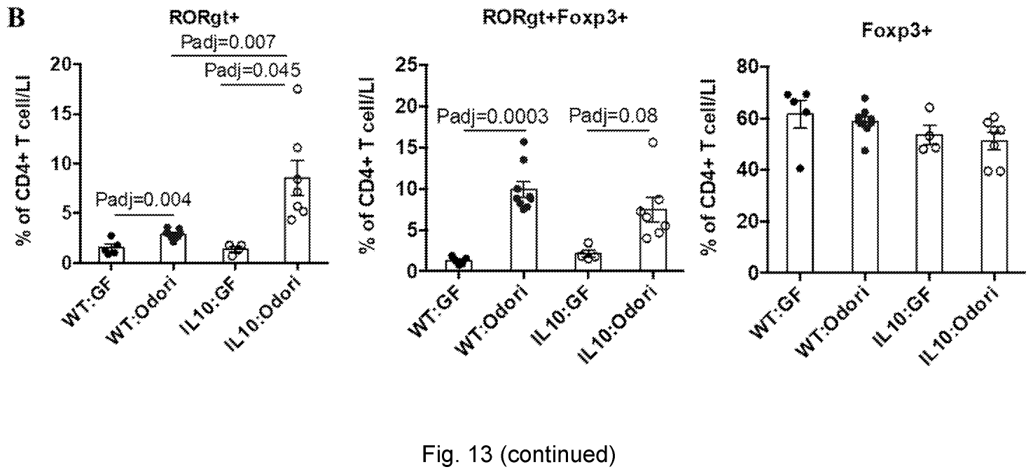

Flow cytometry of live, CD4+ T cells was used to evaluate IL-17 and IFNg expression in large intestine lamina propria cells after 4-hour stimulation with PMA/ionomycin with brefeldin A. (D) Mean percentages are shown for each experimental group, and error bars reflect SEM. P-values indicated, T-test. Data are compiled from two independent experiments. (E) Lamina propria mononuclear cells (LPMCs) were isolated from rectal endoscopic biopsies taken before and 4 weeks after FMT. Flow cytometry of live, CD4+ T cells was used to evaluate Foxp3. Linear regression analyses reveal a significant correlation for Δ Mayo and Δ Foxp3 (P=0.022), as well as, Δ Odoribacter IgA-coated genus and A Foxp3 (P=0.007).(F) While control mice lost weight, O. splanchnicus colonization showed comparatively less weight loss (FIG. 3F, P-value=0.035).

FIG. 4. O. splanchinicus induced IL-10 limits T cell inflammation and colitis. A. Expression of 1110 in colonic tissue in germ free (GF) mice on mice colonized with Odoribacter (odori). B. Flow cytometry of lamina propria mononuclear cells from IL-10 deficient GF or Odoribacter colonized mice. RORgt and Foxp3 transcription factor staining of CD4+ T cells are shown and bar graphs quantitate data from multiple experiments. C. The ratio of RORgt+Foxp3+ CD4+ T cell/RORgt+ CD4+ T cells is show for WT or IL-10 deficient mice colonized with Odoribacter. D, E. Survival (D) or Lipocalin-2 (Lcn-2) levels (E) in WT or IL10-deficient GF or Odoribacter (Odori) mono-colonized mice is shown following 7 day treatment with dextran sodium sulfate (DSS). F. Body weight of WT or IL-10 deficient mice colonized with Odoribacter following 7 day treatment with dextran sodium sulfate. G. The ratio of RORgt+Foxp3+ CD4+ T cell/RORgt+ CD4+ T cells or IL-17+ CD4+ T cells is show for WT or IL-10 deficient mice colonized with Odoribacter following 7 day treatment with dextran sodium sulfate. H. Body weight of RAG-deficient mice colonized with Odoribacter following 7 day treatment with dextran sodium sulfate.

FIG. 5. O. splanchnicus production of butyrate mediates protection against colitis. A. Fecal abundance of SCFA-coA in mice colonized with Odoribacter (Odori), Alistipes, or germ free (GF). B. Body weight of WT mice mono-colonized with Alistipes following 7 day treatment with dextran sodium sulfate. C, D. Body weight (C) Lipocalin-2 (Lcn-2) levels (D) and of Gpr109a-deficient or heterozygous littermate control mice colonized with Odoribacter or control following 7 day treatment with dextran sodium sulfate. P-value between groups is indicated.

FIG. 6. Transferable microbes in FMT non-responders. (A) Boxplot comparing the median relative abundance of the CTM in DON, PRE and WK4 post FMT by FMT clinical response. P-values indicated, Wilcoxon-paired test followed by Bonferroni multiple comparison correction test. (B) Correlation between Δ CTM and UC clinical improvement index (Δ Mayo). (C) Boxplot comparing the median relative abundance of the responders CTM in responders or non-responders DON, PRE and WK4 post FMT. P values indicated, Wilcoxon-paired test followed by Bonferroni multiple comparison correction test. (D) Venn diagram depicting the core shared microbes between non-responders: DON, PRE and at WK4 post FMT. Microbial cut off for prevalence was defined as ≥50% and relative abundance as ≥0.1%. (E) Heatmap depicting the relative abundance of the 15 species defined as being part of the core shared microbes between non-responders' DON and WK4 post FMT. (F) Boxplot comparing the median relative abundance of non-responders CTM in in responders or non-responders DON, PRE and WK4 post FMT. P-values indicated, Wilcoxon-paired test followed by Bonferroni multiple comparison correction test.

FIG. 7. Diversity metrics of IgA-coated microbiota. (A) Boxplot depicting the percentage of IgA-coated bacteria in fecal homogenate of DON, PRE and WK4 groups. P-values indicated, Wilcoxon-paired test followed by Bonferroni multiple comparison correction test. Boxplot comparing WK4 median relative abundance of TIM between responders and non-responders. P-value, Mann-Whitney non-parametric test. (B) Alpha diversity analysis is shown. Boxplot comparing DON, PRE and WK4 post FMT median Shannon index. P-values indicated, Wilcoxon-paired test followed by Bonferroni multiple comparison correction test. Boxplot comparing WK4 median Shannon index between responders and non-responders. P-values indicated, Wilcoxon test. (C) Principal coordinate analysis plot is shown using Unweighted Unifrac and stratified by donor (DON), recipient pre (PRE) and at week 4 (WK4) post FMT. P-value is shown, Monte Carlo, PERMANOVA. (D) Venn diagram depicting the number of bacterial species unique to a single variable (DON, PRE or WK4) or shared by two, or all three variables. Microbial cut off for prevalence was defined as ≥50% and relative abundance as ≥0.1%. In total 1002 species did not enter any of the core groups shown in the Venn diagram. (E) Boxplot comparing the median relative abundance of the TIM in DON, PRE and WK4 post FMT. P values indicated, Wilcoxon-paired test followed by Bonferroni multiple comparison correction test.

FIG. 8. IgA-coating of transferable microbes in gnotobiotic mice colonized with human FMT donor is T-cell independent. (A) The IgA-sequencing strategy is shown. Fecal homogenate was filtered, blocked, and stained with cell-permeable DNA dye Syto BC and anti-IgA antibody. Samples were gated on the basis of forward scatter (FSC) and side scatter (SSC). Heterozygous (Het) mice, TCRβδ^{-/-} mice and unstained. (B) Bar plot depicts fecal bacterial genera detected in human donor and recipient's mice. (C) Principal coordinate analysis plot is shown using Weighted UniFrac distance and stratified by IgA+ and IgA− sample groups. P-value is shown, Monte Carlo, PERMANOVA.

FIG. 9. Odoribacter is only IgA-coated microbe that correlates with clinical response.

FIG. 10. Humanized Mouse Model: TIMs induce IgA in a T cell independent manner. (A) Bar plot depicts the fecal bacterial genera composition detected in human donor and TCRβδ^{-/-} recipient's mice. (B) Boxplot displays the significant differential abundant bacterial genera detected in TCRβδ^{-/-} and het recipient's mice when IgA+ and IgA− bacteria were compared. Kruskal-Wallis, FDR− adjusted P-value <0.05. (C) Principal coordinate analysis plot from fecal samples is shown using Weighted UniFrac and stratified by recipient' mice genotype: Heterozygous (Het) and TCRβδ^{-/-} (TCR). P-value is shown, Monte Carlo, PERMANOVA.

FIG. 11. Circular genome map of UC-derived Odoribacter splanchnicus isolate. The genome of UC-derived Odoribacter splanchnicus isolate was sequenced using PacBio and assembled de novo into 7 contigs. The complete circular genome was 4,712,004 bp in length with a GC content of 43.48% and encoded 4,069 CDS. The genome of UC-derived Odoribacter splanchnicus was annotated using RAST and visualized using the GCView Server. Comparative genomic analyses were performed for UC-derived Odoribacter splanchnicus (blue, outer circle) using Odoribacter splanchnicus DSM (brown) as a reference genome.

FIG. 12. UC-derived O. splanchinicus induces iTregs and alleviate T cell colitis. (A) Mean log10 number of the 16S rRNA gene detected in fecal samples of WT and IL10−/− mice colonized with 2×10⁹ CFU of UC-derived O. splanchnicus and analyzed on day 7-10 post-colonization. (B) Lamina propria mononuclear cells (LPMCs) were isolated from rectal endoscopic biopsies taken before and 4 weeks after FMT. Flow cytometry of live, CD4+ T cells was used to evaluate Foxp3. Linear regression analyses reveal a significant correlation for WK4 Odoribacter IgA-coated genus and Δ Foxp3 (P=0.007). (C) Lamina propria mononuclear cells (LPMCs) were isolated from rectal endoscopic biopsies taken before and 4 weeks after FMT. LPMCs were stimulated with phorbol myristate acetate/ionomycin for 4 hours for intracellular cytokine staining. The percentage of total CD4+ T cells expressing the designated transcription or cytokine were used. Linear regression analyses Δ transcription factor/cytokine and Δ Mayo is shown.

FIG. 13. DSS in UC-derived O. splanchinicus mono-colonization of WT and IL10-deficient mice. (A) WT and IL10−/− mice colonized with 2×10⁹ CFU of UC-derived O. splanchnicus for 7 days followed by 6 days treatment with 2% DSS. (B) Flow cytometry of live, CD4+ T cells was used to evaluate RORgt and Foxp3 expression in large intestine lamina propria. Mean percentages are shown for each experimental group, and error bars reflect SEM. P values indicated, T-test followed by Bonferroni multiple testing correction. Data are compiled from two out of three independent experiments.

### DESCRIPTION OF THE DISCLOSURE

This disclosure provides compositions and methods for treatment of inflammatory bowel diseases, such as UC. The compositions comprise one or more types of bacteria that are identified in this disclosure as core transferable microbiota. The disclosure also provides a method for the treatment of UC comprising administering to an individual in need of treatment a composition comprising the core transferable bacteria, wherein administration of the bacteria results in alleviation of one or more symptoms of inflammatory bowel disease. In an embodiment, administration of the bacteria results in reducing the severity of the IBD. In an embodiment, administration of the bacteria results in reducing or eliminating all of the symptoms of the inflammatory bowel disease.

This disclosure provides identification of transferable microbiota and IgA− coated microbiota in recipients of fecal transplants for ulcerative colitis that had clinical improvement. Further, a role for an Odoribacter strain in regulating the induction of Th17 and RORgt+ Tregs cells has been identified.

Suitable bacteria for the present compositions may be isolated from fecal material. The process of preparing may include collecting a fecal sample from a donor, processing the fecal sample to isolate and purify the selected bacteria, optionally lyophilizing or “freeze-drying” the processed fecal sample (or otherwise converting the processed fecal sample from a liquid to a solid), adding one or more additives and/or excipients, and forming a desired form (such as oral form or form deliverable via enema) of the composition. The isolated, purified bacteria may be in a lyophilized or freeze dried form by itself or the composition may comprise materials and additives suitable for a form for oral administration (e.g., a tablet, capsule, liquid preparation, or the like). Other forms for administration via other routes (e.g., rectal) may alternatively or additionally be used. Methods of isolation and purification of microbiota from fecal material are described in U.S. Pat. No. 10,391,064, the description of which methods is incorporated herein by reference as an example or isolation and purification methods.

In an embodiment, fecal bacteria that are enriched in the IgA coated fraction (such that the ratio of (bacteria abundance in the IgA+ fraction)/(bacteria abundance in the IgA− fraction)>1) may be used for the present compositions. Such bacteria may be the only bacteria present in the composition. An example of such bacteria is Odoribacter and Alistipes. While not intending to be limited by the consideration, it is considered that Alistipes may act via T cell mediated manner, whereas Odoribacter may be T cell dependent and/or T independent (such as seen in a DSS model). This may be related to metabolic production of short chain fatty acids by Odoribacter.

In an embodiment, the disclosure provides a pharmaceutical composition comprising, consisting essentially of, or consisting of bacteria obtained from a fecal source that are IgA coated.

In an embodiment, the disclosure provides a pharmaceutical composition comprising, consisting essentially of, or consisting of bacteria of the genus Streptococcus, Ruminococcus, Roseburia, Odoribacter, Lachnospiraceae, Eubacterium, Coprococcus, Collinsella, Catenibacterium, Bilophila, Bifidobacterium, Barnesiella, Bacteroides, and Alistipes.

In an embodiment, the pharmaceutical composition comprises, consists essentially of, or consists of bacteria from the genus Odoribacter and/or Alistipes.

In an embodiment, the disclosure provides a pharmaceutical composition comprising, consisting essentially of, or consisting of Streptococcus salivarius, Ruminococcus bromii, Roseburia inulinivorans, Roseburia intestinalis, Roseburia hominis, Odoribacter splanchnicus, Lachnospiraceae bacterium 51 63FAA, Eubacterium ramulus, Eubacterium eligens, Eubacterium biforme, Coprococcus catus, Collinsella aerofaciens, Catenibacterium mitsuokai, Bilophila unclassified, Bifidobacterium longum, Bifidobacterium adolescentis, Barnesiella intestinihominis, Bacteroides massiliensis, Bacteroides dorei, and/or Alistipes shahii.

In an embodiment, this disclosure provides a pharmaceutical composition comprising, or consisting essentially of Odoribacter splanchnicus.

In an embodiment, this disclosure provides a pharmaceutical composition comprising, or consisting essentially of Alistipes shahii.

In an embodiment, this disclosure provides a pharmaceutical composition comprising or consisting essentially of Odoribacter splanchnicus and Alistipes shahii.

In an embodiment, the disclosure provides a pharmaceutical composition comprising, or consisting essentially of bacteria of the genus Sutterella, Roseburia, Parabacteroides, Eubacterium, Coprococcus, Catenibacterium, Bifidobacterium, Barnesiella, Bacteroides, and/or Alistipes.

In an embodiment, the disclosure provides a pharmaceutical composition comprising, consisting essentially of, or consisting of Sutterella wadsworthensis, Roseburia inulinivorans, Roseburia intestinalis, Parabacteroides merdae, Parabacteroides distasinis, Eubacterium ramulus, Eubacterium eligens, Coprococcus catus, Catenibacterium mitsuokai, Bifidobacterium bifidum, Bifidobacterium adolescentis, Barnesiella intestinihominis, Bacteroides stercoris, Bacteroides massiliensis, Bacteroides dorei, Bacteroides caccae, Alistipes shahii and/or Alistipes putredinis.

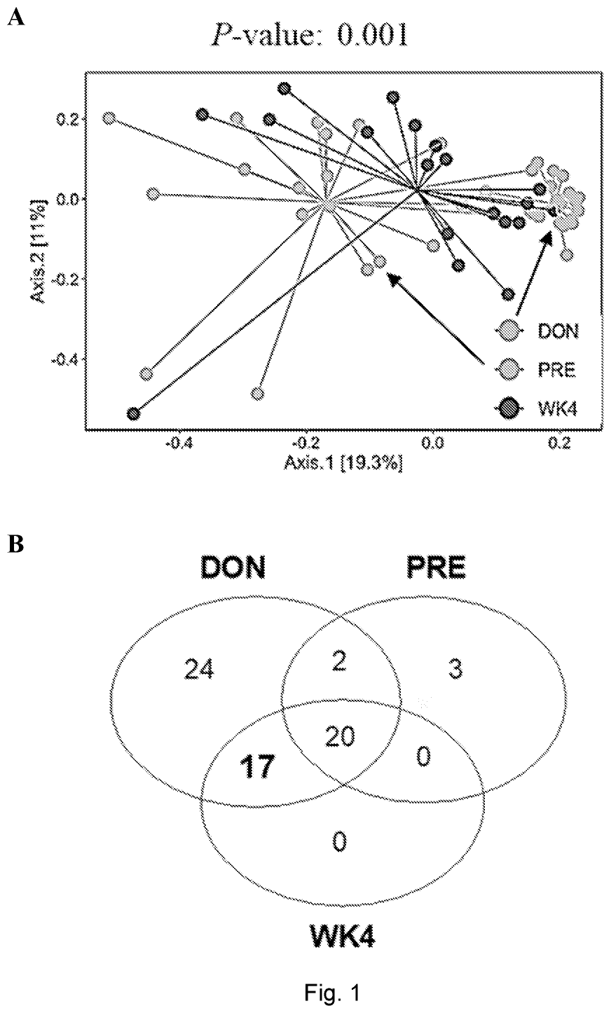

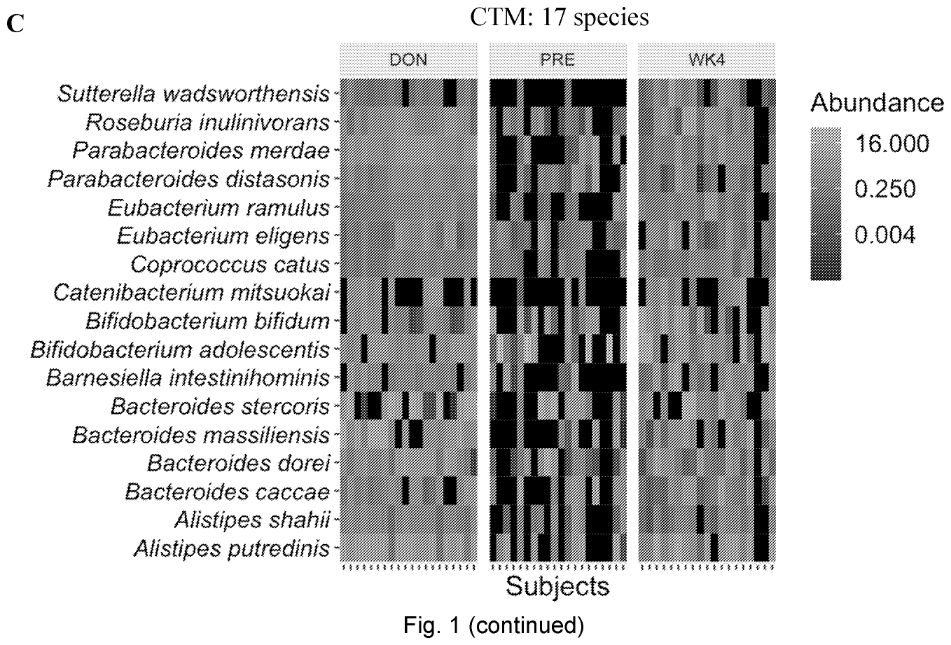

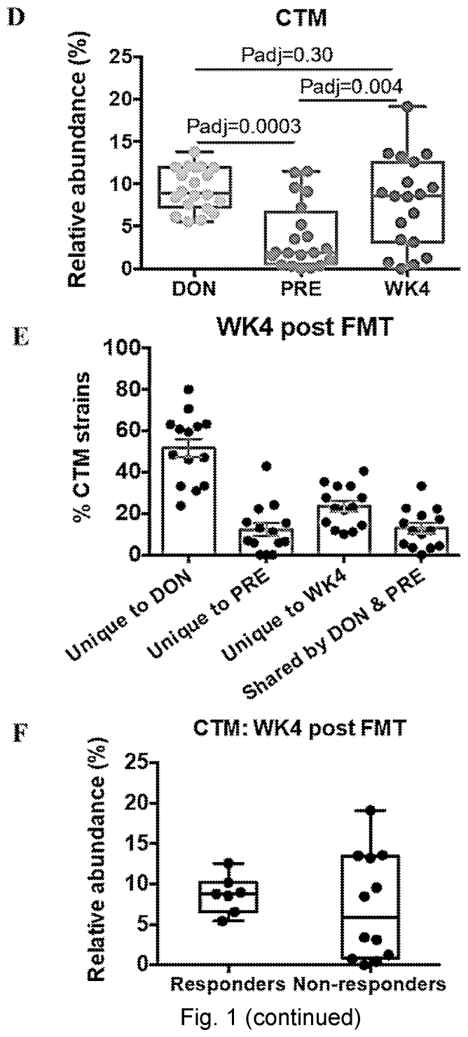

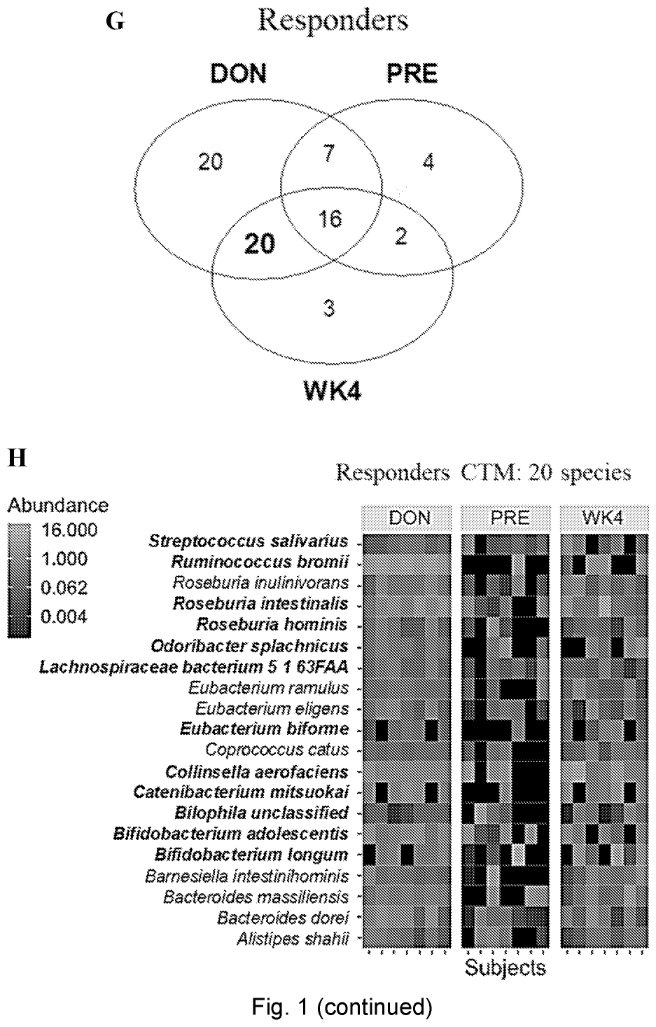

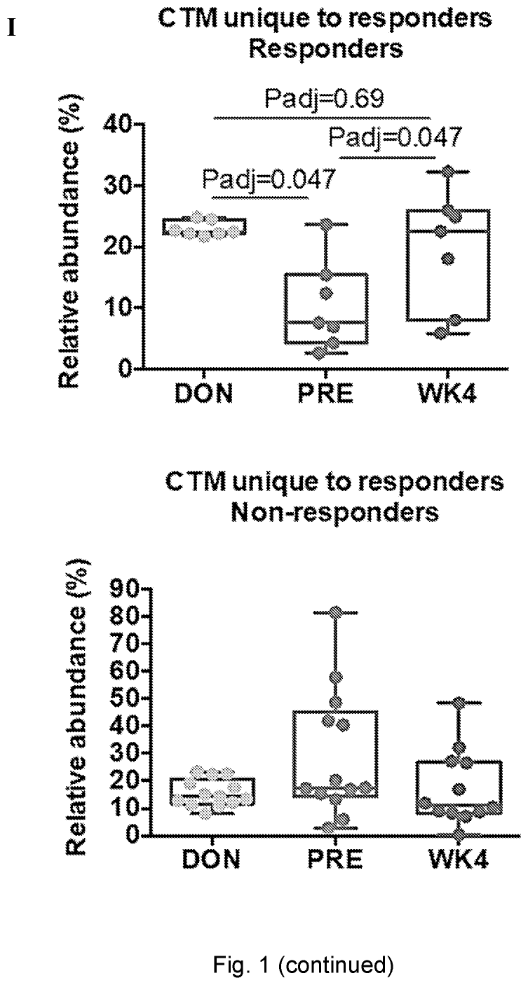

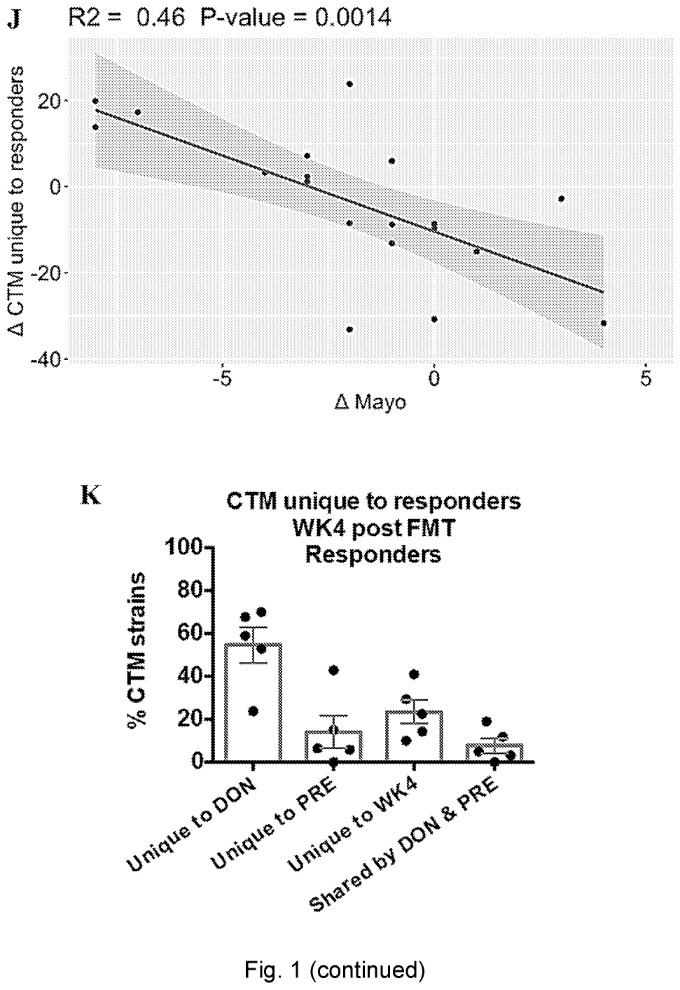

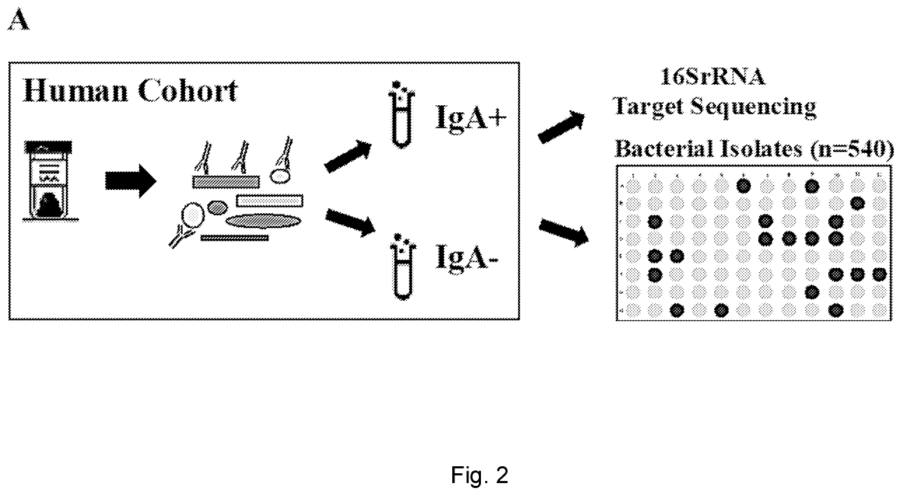

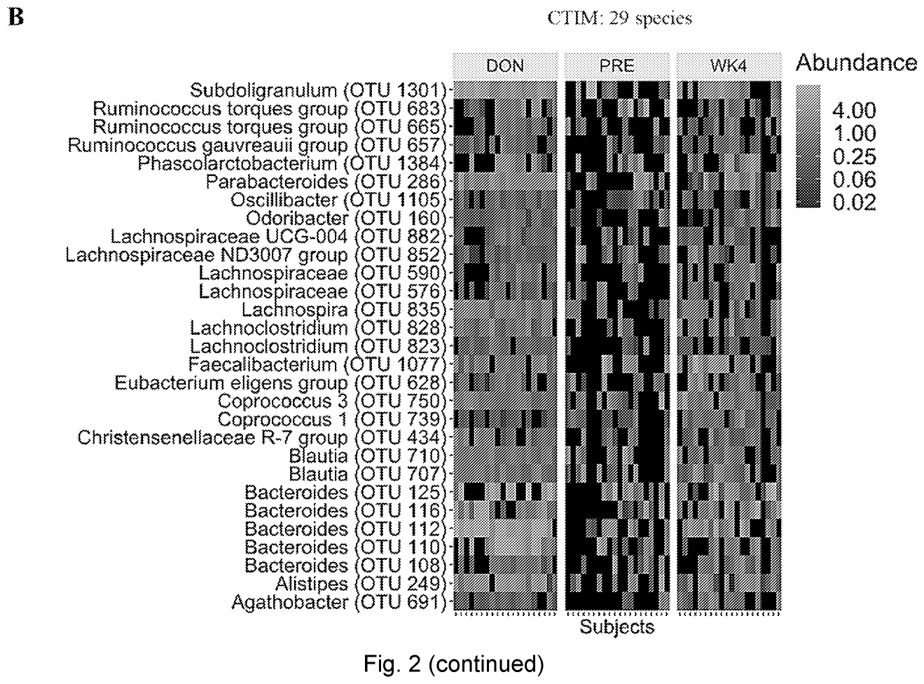

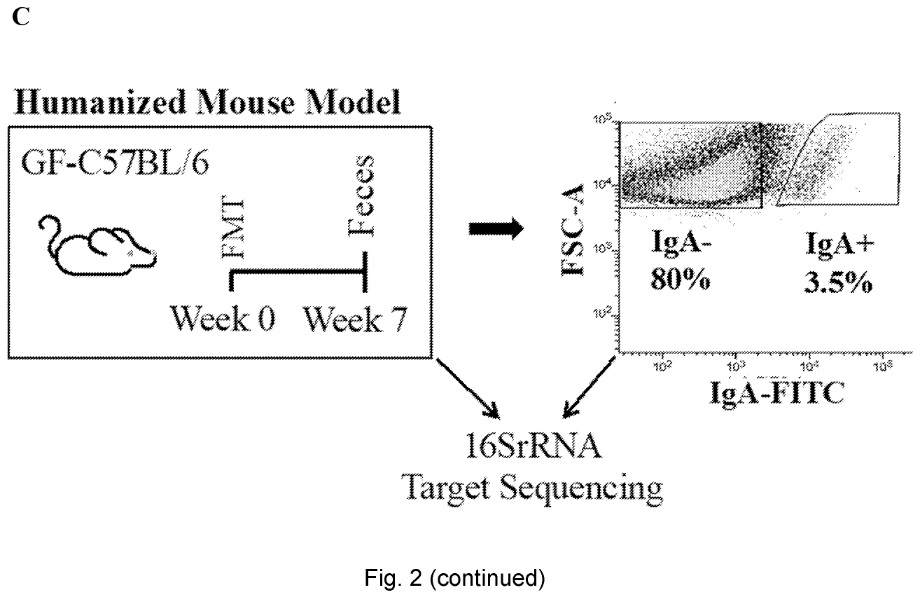

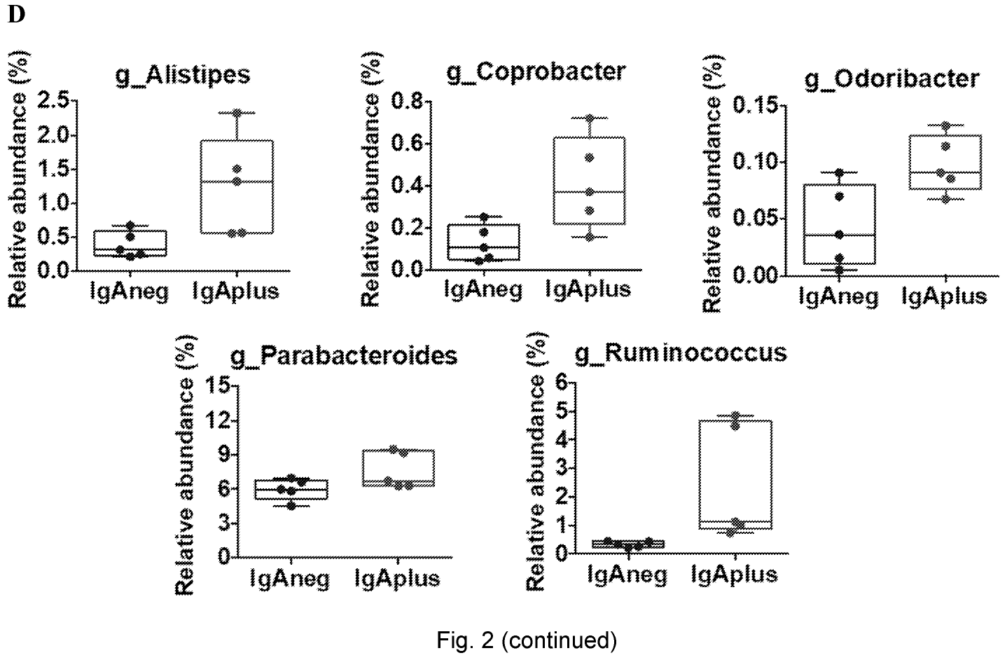

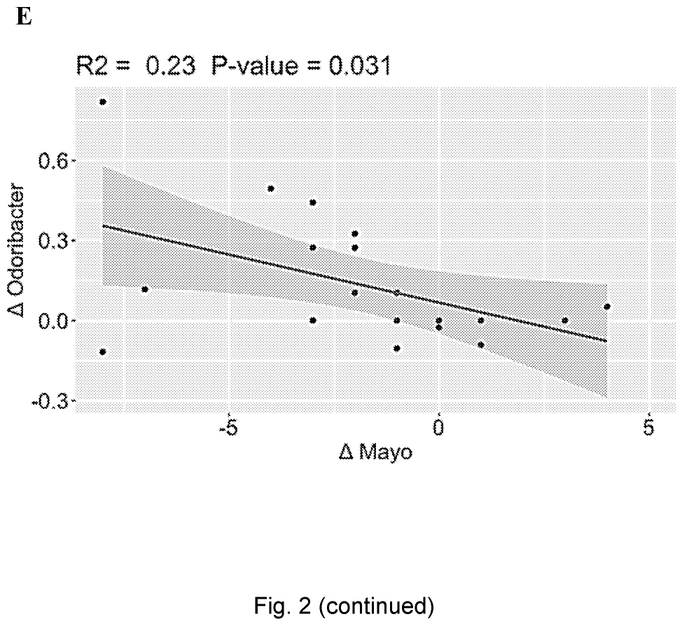

In an embodiment, the present compositions may comprise, consist essentially of, or consist of one or more of the bacteria listed in any of the figures. For example, the present compositions may comprise, consist essentially of, or consist of one or more of the bacteria listed in any one of FIGS. 1C, 1H, and 2B.

The amount of bacteria (individual type or all types) per dose may be 100 million to 1 billion or 100 million to 10 billion and all values and ranges therebetween. In one embodiment, a dose may have more than 1 billion bacteria (individual type or all types). A dose may be a tablet, capsule, or a specified amount of the formulation in any form. In various embodiments, the bacteria (individual type or all types) per dose may be 100, 200, 300, 400, 500, 600, 700, 800, 900 million or 1 billion, 2 billion, 3, billion, 4 billion, 5 billion, 6 billion, 7 billion, 8 billion, 8 billion, 9 billion, 10 billion, etc.

The composition comprising, consisting essentially of or consisting of the bacteria as described herein may optionally be administered with IL-10, which may be present in the same composition as the bacteria or may be present as a separate composition, and may be administered at the same time or different time, by the same route or different route. The amount of each of IL-10 per dose may be 10 micrograms per kilogram to 1 mg per kilogram/body weight and all values and ranges therebetween. For example, the amount may be 50 to 200 micrograms per kilogram.

In embodiments, the compositions may optionally comprise a fiber component. The fibers may be soluble fibers or insoluble fibers. Examples of fibers include fibers originating from seeds (e.g. linseeds and psyllium seeds) or from nuts (such as walnuts, coconuts, almonds), or any other part of the plant may be used. In embodiments, the fiber may be oat fiber, wheat fiber, rye fiber, chia fiber, corn fiber, barley fiber, potato fiber, fruit fiber, vegetable fiber, cereal fiber and fiber from algae. The amount of fiber in the formulation can be from 5 g to 30 g per day or per dose. It may be provided in a single or multiple doses. The fiber may be provided in the same or separate compositions.

In some embodiments, the present disclosure provides a method for treatment of ulcerative colitis comprising administering to an individual in need of treatment a composition comprising, consisting essentially of, or consisting of one or more of Odoribacter species and Alistipes species. In embodiments, the Odoribacter bacteria may be Odoribacter splanchnicus, and the Alistipes bacteria may be Alistipes shahii. The method may optionally further comprise administering to the individual one or more of Streptococcus salivarius, Ruminococcus bromii, Roseburia intestinalis, Roseburia hominis, Lachospiraceae bacterium 5 1 63FAA, Eubacterium biforme, Collinsella aerofaciens, Catenibacterium mitsuokai, Bilophila, Bifidobacterium adolescentis, and Bifidobacterium longum. In an embodiment the method comprises administering to an individual in need of treatment one or more of Odoribacter splanchnicus, Alistipes shahii, Streptococcus salivarius, Ruminococcus bromii, Roseburia intestinalis, Roseburia hominis, Lachospiraceae bacterium 5 1 63FAA, Eubacterium biforme, Collinsella aerofaciens, Catenibacterium mitsuokai, Bilophila, Bifidobacterium adolescentis, and Bifidobacterium longum. In embodiments, the method may further comprise administering to the individual a fiber component, which may be soluble or insoluble fiber.

The pharmaceutical composition can be formulated for oral administration. The present oral compositions may be in the form of a chewable formulation, a dissolving or dissolved formulation, an encapsulated/coated formulation, a multi-layered lozenges (to separate active ingredients and/or active ingredients and excipients), a slow release/timed release formulation, or other forms suitable for oral delivery known in the art. It may be in the form of a tablet, lozenges, pill, capsule, drops or the like. The formulations may also be present as encapsulated or incorporated into micelles, liposomes, cyclodextrins, polymers and the like. The pharmaceutical compositions, including pediatric formulations, may be flavored (e.g. fruit flavored, such as cherry, strawberry, blueberry, etc.) and may be in a variety of shapes or colors.

The pharmaceutically acceptable compositions may comprise a therapeutically-effective amount of one or more of the bacterial species as described above. The compositions may be formulated together with one or more pharmaceutically acceptable excipients. The present disclosure provides pharmaceutically acceptable compositions which comprise a therapeutically-effective amount of one or more of the bacteria species as described above, formulated together with one or more pharmaceutically acceptable excipients and optionally other therapeutically effective medications known in the art allowing for but not limited to combination therapies to improve overall efficacy of each individual therapeutic or to limit the concentration of either therapeutic to avoid side effects and maintain efficacy. The active ingredient and excipient(s) may be formulated into compositions and dosage forms according to methods known in the art. The pharmaceutical compositions of the present invention may be specially formulated for administration in solid or liquid form, including those adapted for the following: oral administration, for example, tablets, capsules, powders, granules, and aqueous or non-aqueous solutions or suspensions, drenches, or syrups, frozen or freeze-dried forms; or intrarectally, for example, as a pessary, cream or foam. The pharmaceutical compositions may be present in a powdered form, freeze-dried form, or lyophilized form.

A therapeutically effective amount of the pharmaceutical composition of the present invention is sufficient to promote the health of the gastrointestinal tract, or to alleviate one or more symptoms of UC. The dosage of active ingredient(s) may vary, depending on the individual subject. The dosage may be adjusted based on the subject's weight, the age and health of the subject and this is well within the purview of those skilled in the art.

The term “therapeutically effective amount” as used herein refers to an amount of an agent sufficient to achieve, in a single or multiple doses, the intended purpose of treatment. Treatment does not have to lead to complete cure, although it may. Treatment can mean alleviation of one or more of the symptoms or markers of the indication. The exact amount desired or required will vary depending on the particular compound or composition used, its mode of administration, patient specifics and the like. Appropriate effective amount can be determined by one of ordinary skill in the art informed by the instant disclosure using only routine experimentation. Within the meaning of the disclosure, “treatment” also includes relapse, or prophylaxis as well as the alleviation of acute or chronic signs, symptoms and/or malfunctions associated with the indication. Treatment can be orientated symptomatically, for example, to suppress symptoms. It can be effected over a short period, over a medium term, or can be a long-term treatment, such as, for example within the context of a maintenance therapy. Administrations may be intermittent, periodic, or continuous.

The phrase “pharmaceutically acceptable” is employed herein to refer to those compounds, materials, compositions, and/or dosage forms which are suitable for use in contact with the tissues of the subject with toxicity, irritation, allergic response, or other problems or complications, commensurate with a reasonable benefit/risk ratio.

The phrase “excipient” in reference to pharmaceutical compositions refers to a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject. Each excipient should be “acceptable” in the sense of being compatible with the other ingredients of the formulation and not injurious to the subject. Some examples of materials which can serve as pharmaceutically-acceptable excipients include sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations. If desired, certain sweetening and/or flavoring and/or coloring agents may be added. Other suitable excipients can be found in standard pharmaceutical texts, e.g. in “Remington's Pharmaceutical Sciences”, The Science and Practice of Pharmacy, 19th Ed. Mack Publishing Company, Easton, Pa., (1995).

Excipients are added to the composition for a variety of purposes. Diluents increase the bulk of a solid pharmaceutical composition, and may make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate and starch.

In liquid pharmaceutical compositions of the present invention, the bacterial species and any other solid excipients are dissolved or suspended in a liquid carrier such as water, water-for-injection, vegetable oil, alcohol, polyethylene glycol, propylene glycol or glycerin. Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol and cetyl alcohol. Liquid pharmaceutical compositions of the present invention may also contain a viscosity enhancing agent to improve the mouth feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth and xanthan gum. A liquid composition may also contain a buffer such as gluconic acid, lactic acid, citric acid or acetic acid, sodium gluconate, sodium lactate, sodium citrate or sodium acetate.

Sweetening agents such as sorbitol, saccharin, sodium saccharin, sucrose, aspartame, fructose, mannitol and invert sugar may be added to improve the taste. Flavoring agents and flavor enhancers may make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol and tartaric acid. Preservatives and chelating agents such as alcohol, sodium benzoate, butylated hydroxy toluene, butylated hydroxyanisole and ethylenediamine tetraacetic acid may be added at levels safe for ingestion to improve storage stability.

The individual or subject that may be administered a composition of the present disclosure may be any animal, including human and non-human animals. Non-human animals includes all vertebrates, e.g., mammals and non-mammals, such as non-human primates, sheep, dogs, cats, cattle including cows, pigs, horses, poultry, chickens, and the like. The subjects, such as human subjects may suffering from UC or at risk for UC. The subject is generally diagnosed with an intestinal bowel disease, such as UC by skilled artisans, such as a medical practitioner. A Mayo Clinical Score, also referred to as Mayo Score may be used in the diagnosis. This score refers to an index system for assessing the severity of a ulcerative colitis disease condition. See Schoeder et al., N Engl J Med 1987, 317:1625-1629. The Mayo Clinic score may range from 0-12, with sub-scores of 0-3, where the higher scores indicate more severe disease. Sub-scores may be rated for stool frequency, rectal bleeding, mucosal appearance at endoscopy, and physician's global assessment (PGA). In an embodiment, the individual in need of treatment has a Mayo score of less than 4. In an embodiment, the individual in need of treatment has a Mayo score of at least 4 prior to treatment. In embodiments, the individual may have a Mayo score of 4, 5, 6, 7, 8, 9, 10 or any range between 3 and 10, such as 4 to 10, 4 to 9, 5 to 10, 5 to 8, or 6 to 8.

Another indicator of UC is “ulcerative colitis endoscopic index of severity” and refers to an index for assessing endoscopic disease activity. The index assesses three criteria, including vascular pattern, bleeding, and erosions and ulcers (See Travis et al., Gut 2012, 61(4):535-542). A higher score reflects increased disease severity.

In general, the common symptoms of ulcerative colitis are abdominal pain, and bloody stools, and diarrhea. This may be accompanied by fever, rectal bleeding, fatigue, anemia, loss of appetite, weight loss, and fluids and nutrient loss.

The term “Shannon Diversity Index” refers to a diversity index that accounts for abundance and evenness of species present in a given community (See Shannon and Weaver, (1949) The mathematical theory of communication. The University of Illinois Press, Urbana. 117 pp). Higher values indicate diverse and equally distributed communities, and a value of 0 indicates only one species is present in a given community.

The present compositions and methods relate to use of fecal matter transplant therapies (FMT), which involves using intestinal bacteria from a healthy or desired individual's fecal matter and then processing and transferring that bacteria to the infected patient directly. The fecal matter may be processed to extract the bacteria. The transplantation of the fecal matter is generally carried out by colonoscopy, endoscopy, sigmoidoscopy, or enema. FMT may also be carried out by using frozen or freeze-dried fecal microbiota administered in pill form.

The appropriate dosage and treatment regimen of the probiotic compositions may be determined or recommended by a clinician or nutritionist. In general, one or more doses may be administered per day for a day, week, and month or longer if needed. For example, a dose may be administered every day for 1 week. In embodiments the compositions may be provided via enema or oral route. The treatment may be carried out as long as needed for the symptoms to alleviate or may be used as a maintenance therapy.

Some embodiments of the present disclosure are provided below as Examples:

Example 1. A pharmaceutical composition comprising, consisting essentially of, or consisting of bacteria from the genus Odoribacter and/or Alistipes.

Example 1a. The composition of example 1, wherein the only bacteria present in the composition are Odoribacter, Alistipes or both.

Example 2. A pharmaceutical composition comprising, consisting essentially of, or consisting of transferrable, immune reactive microbiota obtained from fecal material.

Example 2a. The composition of example 2, wherein the microbiota is capable of inducing IgA reactivity.

Example 2b. The composition of example 2 or 2a, wherein the microbiota comprises, consists essentially of, or consists of bacteria of genus Odoribacter.

Example 2b1. The composition of example 2b, wherein the bacteria is Odoribacter splanchnicus.

Example 2c. The composition of example 2 or 2a, wherein the microbiota comprises, consists essentially of, or consists of bacteria of genus Alistipes.

Example 2c1. The composition of example 2c, wherein the bacteria is Alistipes shahii.

Example 3. A method to treat inflammatory bowel disease (IBD) comprising administering to an individual in need of treatment a composition of any of the preceding examples.

Example 3a. The method of example 3, further comprising administering to the individual IL-10.

Example 3b. The method of example 3 or 3a, wherein the IBD is uncreative colitis.

The following examples further describe the invention. These and other examples in this disclosure are illustrative and not restrictive.

### EXAMPLE 1

IBD disease activity scores were used to define clinical response. Metagenomic sequencing of donor, recipient, and 4 week post-FMT fecal samples was performed to define the CTM. Strain level transferability was defined using the StrainFinder algorithm. To define the transferable immune-reactive microbiota (TIM), IgA-seq was performed on donor, recipient, and 4 week post-FMT fecal samples. TIM strains were isolated from fecal samples and gnotobiotic mouse models were used to evaluate their impact on mucosal immunity and mouse models of colitis.

Here, we defined a CTM associated with clinical response to FMT for UC. Strain level tracking of the CTM confirmed that clinical response correlated with strain transferability. In addition, we defined a core TIM by IgA-seq that correlated with clinical response. In humanized mouse models, these TIM were found to induce IgA in a T cell independent manner. Colonization of germ-free mice with a core TIM strain of Odoribacter induced IL-10-dependent, RORgt+/Foxp3+ iTreg cells and reduced the severity of transfer T cell colitis in mono-colonized RAG−/− mice.

Our data highlight an immune-reactive, core transferable microbiota in responders to FMT for UC. Using pre-clinical mouse models of colitis, we define the mechanistic impact of these TIM in shaping mucosal immunity and guiding the response to UC. This work provides a framework for rational selection of TIM for microbial-therapy in IBD.

### EXAMPLE 2

This examples describes identification of core transferable immune-reactive microbiota (TIM).

### Methods

### Study Population

20 individuals with UC received FMT using two-healthy donor fecal microbiota preparation (FMP). Study participants were prospectively followed and data were collected longitudinally. Briefly, participants were age ≥18 years old with biopsy-proven UC and active disease defined as Mayo score ≥3 along with an endoscopic subscore ≥1. At screening and week 4 post-FMT, fecal samples as well as rectal mucosal biopsies were collected for microbiome and immune analyses, respectively. Patients with antibiotic use 3 months before study enrollment were excluded from the study. Clinical responders were defined as A Mayo score ≥3 and a bleeding subscore <1.

### Metagenomic Analysis

DNA from fecal samples was isolated using the MagAttract PowerMicrobiome DNA/RNA kit with glass beads (Qiagen, Germany). Metagenomic libraries were prepared using the NEBNext Ultra II for DNA Library Prep kit (New England Biolabs, Ipswich, MA) following the manufacturer's protocol. The DNA library was loaded on an Illumina HiSeq instrument using a 2×150 paired-end configuration in a high output run mode. Sequencing data was processed using Huttenhower lab bioinformatics tools (huttenhower.sph.harvard.edu) and human contaminant reads removed using KneadData (bitbucket.org/biobakery.kneaddata). To filter out low quality sequence reads, sequences shorter than 50 bp and Illumina adapters reads, Trimmomatic was used as implemented in KneadData. After quality control, samples averaged 135 million reads (mean=135,854,707;

SEM±4,220,417.1). Taxonomic profiling was then determined by MetaPhlAn2 pipeline version 2.7.5. Microbial abundances were calculated with MetaPhlAn2, following Bowtie2 alignment to the MetaPhlAn2 unique marker database.

To analyze microbial strains, metagenomic data was analyzed using StrainFinder pipeline (Smillie et al., Cell Host Microbe 2018;23:229-240 e5). AMPHORA bioinformatics workflow was used to identify single-copy phylogenetic markers in a set of 6,007 dereplicated genomes from PATRIC (patricbrc.org)). Strain Finder aligned the metagenomic reads from a sample against our reference phylogenetic markers and then tabulated the SNPs at every position of the alignment. Strain genotypes and their frequencies were then generated based on maximum likelihood estimates (MLE) approach.

### IgA-16S Seq Analysis

To define the IgA-coated microbiota, fecal homogenates were processed, labeled and sorted. Briefly, feces were homogenized in 5% bovine serum albumin (BSA) diluted in 1× phosphate-buffered saline (PBS) and clarified supernatants were then washed with 5% BSA-PBS followed by 20 minutes incubation with blocking buffer (human samples: 1% mouse serum; mouse samples: 1% rat serum). Samples were then stained with anti-human IgA (IS11-8E10) or anti-mouse IgA (mA-6E1) and sorted on Melody (BD Biosciences).

Human-derived IgA coated (IgA+) and not-coated (IgA−) sorted samples were processed using PowerMag Soil DNA Isolation Kit (MO BIO), following manufacture instructions. The V4 region of 16S rRNA gene was amplified by PCR and sequenced in the Illumina MiSeq platform using the 2×250 bp paired-end protocol and Diversigen. The read pairs were demultiplexed based on their unique molecular barcodes, denoised and merged using DADA2 (Nature Methods 2016; 581-583), and subject to chimera removal using VSEARCH (VSEARCH: a versatile open source tool for metagenomics. PeerJ 2016; 4:e2584.). 16S rRNA gene sequences were clustered into Operational Taxonomic Units (OTUs) at a similarity cutoff value of 97%. Taxonomic identities were then assigned using the scikit-learn classifier to an specific and optimized 16S-V4 of the SILVA Database (Nucleic Acids Res 2013; 41(Database issue): D590-596.)

A rarefied OTU table (sequence depth of 7,684 reads) from the output files generated in the previous steps was used for alpha and beta diversity, as well as taxonomic downstream analyses.

Mouse-derived IgA coated (IgA+) and not-coated (IgA−) bacteria were sorted as described above and samples were processed using DNeasy PowerLyzer PowerSoil kit (Qiagen, Germany), following manufacturer's instructions. The V4 and V5 regions of 16S rRNA gene was amplified by PCR and sequenced in the Illumina MiSeq platform using the 2×250 bp paired-end protocol at Weill Cornell Microbiome Core Facility. 16S bioinformatics pipeline was implemented using USEARCH (version 11.0.667) (Edgar, R.C. et al 2010-Bioinformatics 26(19), 2460-2461). Briefly, raw read pairs were demultiplexed based on the unique molecular barcodes, and reads were merged. Quality filter was applied to the resulting merged reads and then clustered into OTUs (with simultaneous chimera removal). Taxonomic prediction was performed using usearch -sintax, an implementation of the SINTAX algorithm. The RDP (Nucl. Acids Res. 42:D633-D642) 16S training set (version 16) was used as a reference database for classification. A phylogenetic tree was generated from the representative OTU sequences using “usearch -cluster_aggd”. A rarefied OTU table (sequence depth of 32,903 reads) from the output files generated in the previous steps was used for alpha and beta diversity, as well as taxonomic downstream analyses.

UC-derived O. splanchnicus Genome Sequencing

Whole genome sequencing was performed using PacBio Sequel v3 SMRTcell. Raw sequence data were corrected, trimmed, and assembled using Canu default settings, with a genome size of 4.8 m being specified (Genome Res. 2017, doi:10.1101/gr.215087.116). Long reads were assembled de novo into 6 contigs using minimap2 (v.2.17-r941) (Bioinformatics 2018, 34(18), 3094-3100). Gene prediction and annotation were carried out using RAST (The RAST Server: Rapid annotations using subsystems technology. BMC Genomics 9, 75 (2008)), incorporating the Glimmer algorithm (Identifying bacterial genes and endosymbiont DNA with Glimmer. Bioinformatics 23, 673-679 (2007)). GCView server was used for genome visualization and comparison (Grin, D. Linke, GCView: The genomic context viewer for protein homology searches. Nucleic Acids Res. 39, W353—W356 (2011)).

### Metabolomics Analysis

To determine short chain fatty acid-CoA fecal abundance, feces were treated with cold 80% methanol for 4 hours at −80° C. Samples were then centrifuged at 14,000 g for 20 min at 4° C. Supernatants were transferred to a sterile 1.5 ml tube and stored at -80° C. Samples were submitted to WCM Proteomics and Metabolomics Core Facility for SCFA- CoA metabolomics analysis by High Performance Liquid Chromatography (HPLC). Relative metabolite quantification was performed based on peak area for each metabolite evaluated and in downstream analyses described as fecal abundance. The loading of each fecal sample in the mass spectrometry instrument was adjusted based on their weight.

### Gnotobiotic and SPF Mice

Germ-free C57BL/6 WT, IL-10^{-/-, Rag}1^{-/-}, TCRβδ^{-/-} and heterozygous mice were bred and maintained at WCM. For mono-colonization experiments, 6- to 8-week-old germ-free mice were gavaged with 2×10⁹ CFU log-phase bacteria grown under anaerobic conditions in Yeast Casitone Fatty Acids Broth with Carbohydrates (YCFAC, Anaerobe Systems). Colonization was confirmed by 16S rRNA qPCR from DNA extracted using feces or cecal content.

SPF C57BL/6 GPR109a^{-/-} and GPR43^{-/-} were obtained and bred and maintained at WCM. SPF C57BL/6 Rag2^{-/-} were bred and maintained at WCM. For colonization of GPR109a^{-/-}, GPR43^{-/-}, het, Rag2^{-/-}and WT mice, 5- to 7 weeks were subjected to one week of antibiotic treatment in the drinking water ad libitum (ampicillin, vancomycin, metronidazole, neomycin). After one day washout, mice were gavaged with media or 2×10⁹ CFU of UC-derived O. splanchnicus. Colonization was confirmed by O. splanchnicus 16S rRNA PCR (F primer: 5′-ATGTAATGATGAGCACTCTAACGG-3′ (SEQ ID NO:1); R primer: 5′-GGCTTTTGAGATTGGCATCC-3′) (SEQ ID NO:2) (J. Tong et al./Anaerobe 17 (2011) 64e68 N PMID: 21439390) using DNA extracted from feces or cecal content.

### Colitis Mouse Models

For induction of chemical colitis, 2% DSS (w/v) (M.W. 40,000-50,000; Affymetrix) was added to drinking water and administered ad libitum for 6 days. On day 6 post-DSS initiation, DSS was replaced with normal drinking water. For T cell transfer colitis, ˜5×10⁵ CD4+ CD45RB^{high} CD25− CD62L− T cells were transferred into Rag2^{-/-} or WT mice. For all colitis model experiments, mice were monitored daily (DSS) or weekly (T cell transfer) for weight loss and survival.

To evaluate the level of intestinal inflammation, feces or cecum content were weighed, homogenized in PBS, and centrifuged at 16,000×g (4° C.) for 5 minutes. Mouse Lipocalin-2 was then measured in the supernatant by sandwich ELISA (R&D Systems).

Quantitative PCR (qPCR)

For IL-10 quantification, large intestine lamina propria mononuclear cells (LPMC) were isolated and stored in RLT buffer (Qiagen, Germany). RNA was extracted and purified using RNeasy Plus Micro Kit (Qiagen, Germany) and quantified by Nanodrop spectrophotometer prior to reverse transcription with qScript cDNA Supermix kit (Quanta Biosciences, Canada). Relative levels of IL-10 (IL10-F: 5′ -GAGAGCTGCAGGGCCCTTTGC-3′ (SEQ ID NO:3), IL10-R: 5′-CTCCCTGGTTTCTCTTCCCAAGACC-3′) (SEQ ID NO:4)were measured in large intestine LPMC and A cycle threshold (Ct) determined using expression of housekeeping gene HPRT.

In order to determine fecal total 16SrRNA gene copies in our mono-colonization models, patient derived O. splanchnicus was grown anaerobically and the DNA was isolated at log-phase. Amplified V4-V5 hypervariable region of the 16S rRNA gene was purified with a QIAprep Spin Miniprep Kit (Qiagen, Valencia, Calif.) and quantified by Qubit fluorometer (ThermoFisher Scientific, Waltham, Mass.) using dsDNA Broad Range Assay kit (Life Technologies Corporation, Carlsbad, Calif.). Quantification of the 16S rRNA target gene was then achieved by 10-fold serial dilutions ranging from 10⁰ to 10⁵ of the O. splanchnicus purified 16SrRNA amplicon. 16SrRNA gene copies were measured by qPCR using the 16SrRNA primers 515F (5′-GTGCCAGCMGCCGCGGTAA-3′) (SEQ ID NO:5)and 926R (5′-CCGYCAATTYMTTTRAGTTT-3′) (SEQ ID NO:6). 16SrRNA standards and fecal samples were run in duplicates and the average of the Ct value was used for calculation of the total 16SrRNA gene copies.

All qPCR assays were performed on an Applied BioSciences Quant Studio 6 (ThermoFisher Scientific, Waltham, Mass.) using PerfeCTa SYBR Green Fast mix, Low ROX (Quanta Biosciences, Canada) according to manufacturer's protocol.

### Cellular Isolation and Intracellular Cytokine Staining

LPMC were isolated from the large intestine. LIVE/DEAD fixable aqua dead cell stain kit (Molecular Probes) was used to select live cells. For analysis related to transcription factors, large intestine LPMC cells were stained with anti-CD3-E780 (eBiosciences 17A2) and anti-CD4-AF700 (eBiosciences GK1.5) before fixation and permeabilization with Cytoperm/Cytofix flowed by intracellular staining against anti-Foxp3-E450 (eBiosciences FJK-165) and anti-RORγt-PE (eBiosciences B2D) as per manufacturer's instructions (eBiosciences). For analysis related to cytokine, large intestine LPMC cells were stimulated with phorbol myristate acetate (PMA) and ionomycin with BD GolgiPlug for 3.5 hours. Following surface-marker staining with anti-CD3-E780 (eBiosciences 17A2) and anti-CD4-AF700 (eBiosciences GK1.5), large intestine LPMC cells were prepared as per manufacturer's instruction with Cytoperm/Cytofix (BD Biosciences) for intracellular cytokine evaluation of IL-17A (eBiosciences 17B7) and IFNγ (eBiosciences XMG1.2).

### Statistics

Microbiome analysis was performed in R studio (Boston, Mass.). Alpha and Beta diversity was calculated using R package ‘phyloseq’ (PLoS One 2013;8:e61217) while plots were constructed in ‘ggplot2’. Principal coordinate plots used the Monte Carlo permutation test to estimate the P values. Venn diagrams were generated using R package ‘ampvis’ (PLoS ONE 10(7): e0132783. doi:10.1371/journal.pone.0132783) based on a patient prevalence cut off of ≥50% and microbial relative abundance cut off of ≥0.1%. For taxonomic analysis, significance of microbial relative abundance was calculated using the nonparametric Mann-Whitney test and P values were adjusted for multiple hypothesis with the false discovery rate algorithm. If paired analyses were applicable, Wilcoxon-paired rank test was used. Hypothesis testing was done using two-sided test as appropriate at a 95% significance level.

Proportion and number of microbial strains belonging to the CTM was determined based on presence/absence of microbial SNPs identified by StrainFinder. CTM-strains were then stratified into four groups: unique to DON (shared by DON and WK4 samples but absent in PRE samples), unique to PRE (shared by PRE and WK4 samples but absent in DON samples), unique to WK4 (detected only in WK4 samples and therefore absent in DON and PRE samples), and shared by DON and PRE (shared by DON, PRE and WK4 samples).

Correlation between two continuous variables (e.g. microbial relative abundance, A microbial relative abundance, Δ Mayo and Δ immune cell %) was determined with linear regression models, where P values indicate the probability that the slope of the regression line is zero.

To evaluate significance of categorical variables, such as mice genotypes (e.g. IL10^{-/-} and WT) and colonization statuses (e.g. GF and O. splanchnicus colonization), in regards to gene expression, large intestine cell population (%), fecal lipocalin-2 abundance, fecal metabolite abundance, was calculated by t test for comparison of two groups and two-way ANOVA for comparison of more than two groups. The significance of survival curves was determined by log-rank test.

### Results

### Core Transferable Microbes (CTM) Define FMT Clinical Response

A critical need for studying and optimizing FMT for treating human disease FMT is to define the characteristics of a core group of transferable donor-derived microbiota called CTM. To determine the CTM in a cohort of patients with active UC, we performed deep shotgun metagenomic sequencing of 60 (20 donor, described as DON; 20 recipients pre-FMT, described as PRE; and 20 at week 4 post-FMT, described as WK4) samples from a previously reported open-label trial of FMT for the treatment of UC. PCoA based on beta diversity at the species-level and calculated using Bray Curtis method, showed that treatment groups (DON, PRE and WK4) had significantly different microbial composition (PERMANOVA P-value=0.001, FIG. 1A). Consistent with our previously reported 16S rRNA analysis, recipients' microbial composition markedly shifted toward that observed in donor. To determine microbial transferability and identify the core bacterial species acquired from donors, a Venn diagram approach was used (FIG. 1B). Based on a patient prevalence cut off of ≥50% and microbial relative abundance cut off of ≥0.1%, 17 bacterial species were found to be part of the CTM (FIG. 1B, C). The CTM median relative abundance post-FMT (WK4) did not differ from that observed in donors (adjusted Wilcoxon-paired P-value=0.30), but was significantly higher than that found in PRE (adjusted Wilcoxon-paired P-value=0.004, FIG. 1D). Since species-level analysis cannot determine if donors and recipients' post FMT share bacterial strains and to evaluate if this CTM reflected the transfer of donor-derived strains, we performed maximum likelihood analysis using Strain Finder bioinformatics tool. A total of 22±2.37 distinct bacterial strains belonging to the CTM were detected in WK4 post-FMT samples of which 52% (SEM±4.46, mean depth of coverage—MDC=5.4±1.04) were found to be unique to donors (FIG. 1E). Collectively, these results provide evidence of a core set of transferrable bacterial strains in patients with active UC.

We next sought to determine the potential relationship between the CTM and clinical response to FMT. In our cohort and similar to data from recent RCTs, the primary endpoint of clinical response was achieved in 35% of participants (defined as a Δ Mayo score ≥3 with a rectal bleeding score ≤1) by week 4 post-FMT. No significant differences were detected in the CTM mean relative abundance when WK4 samples were compared between responders and non-responders (Mann-Whitney P-value=0.59, FIG. 1F) and the change in CTM relative abundance post-FMT (A CTM) did not correlate with the change in Mayo score (Δ Mayo, P-value=0.15, R²=0.12, FIG. 6A). When analyses were performed within FMT clinical responses, we found that CTM composition was mainly enriched in non-responders' patients (FIG. 6B).

To specifically identify the CTM associated with clinical response, Venn diagram analysis was performed within FMT clinical response (responders, FIGS. 1G and non-responders, FIG. 6D-F). Twenty bacterial species were found to be part of the responders CTM (FIG. 1H). Of these 20 species, 12 were unique to responders CTM (CTM^{responders}: species marked in bold, FIG. 1H), and therefore absent in the non-responders CTM (FIG. 6D and E). As such, the relative abundance of the CTM^{responders} was significantly higher in responders WK4 post-FMT (adjusted Wilcoxon-paired P-value=0.047, FIG. 1I), but not in non-responders WK4 post-FMT (adjusted Wilcoxon-paired P-value=0.08, FIG. 1J). Linear correlation analysis (FIG. 1J) for CTM^{responders} species similarly demonstrated that increase in the relative abundance of the CTM^{responders} significantly correlate with decrease in Mayo score (P-value=0.0014, R²=0.46). Strain Finder analysis detected 25 CTM^{responders} strains (SEM±4.1) with 51% (SEM±4.38, MDC=252.6±49.08) unique to donor (FIG. 1K). This analysis highlights the transferability of a core set of bacterial strains that correlate with clinical efficacy of FMT for UC.

IgA-coating of CTM-associated Odoribacter is Associated with Clinical Response

Given the transferability of strains associated with clinical response observed above, we next sought to determine their potential contribution to mucosal immunity by sorting and sequencing IgA-coated microbiota (FIG. 2A). The proportion of fecal bacteria coated with IgA did not change post-FMT (WK4) and did not differ between responders and non-responders (Wilcoxon P-value=0.80, FIG. 7A); however, the IgA-coated community of WK4 post-FMT was more diverse (adjusted Wilcoxon-paired P-value=0.02, FIG. 7B) and higher in responders than non-responders (Wilcoxon P-value=0.039, FIG. 7B). Beta diversity analysis, based on Unweighted UniFrac, revealed that IgA-coated microbial community of FMT-recipients (WK4) markedly shifted toward that observed in DON (PERMANOVA P-value=0.001, FIG. 7C). To define the set of immune reactive bacteria acquired from donors, the Venn diagram approach was applied to the IgA-coated bacterial community using a patient prevalence cut off of ≥50% and microbial relative abundance cut off of ≥0.1%. 29 IgA-coated OTUs were found to be donor-derived (FIG. 2B and 7D). The median relative abundance of these OTUs were significantly higher in responders when compared to non-responders (Mann-Whitney P-value=0.02, FIG. 7E). Of these 29 OTUs, Odoribacter (1 OTU and the only Odoribacter detected in our cohort), Bacteroides (5 OTUs), Coprococcus (2 OTUs), Eubacterium (1 OTU), Lachnospiraceae (4 OTUs) and Ruminococcus (3 OTUs) were the only taxa to overlap with the CTM (FIG. 1H and 2B).

To confirm the transferability of these IgA-coated taxa in a model system of FMT, we transferred donor material into germ free C57BL/6 mice, sorted and sequenced IgA-coated microbiota (FIGS. 2C and 8A). 16S rRNA sequencing was performed to validate microbial engraftment of donor material (FIG. 8B). The IgA-coated community was significantly different from that of IgA− (FIG. 8C, PERMANOVA P-value=0.007). Six donor-derived genera were significantly enriched in IgA-coated community compared to IgA− (Mann-Whitney, FDR-adjusted P-value≤0.1, FIG. 2D), of which, species from the Alistipes, Odoribacter, and Ruminococcus genera were also members of the CTM (FIG. 1H). Of these four donor-derived genera, only the relative abundance of Odoribacter WK4 post-FMT (FIG. 9) and its increase post-FMT (Δ Odoribacter, FIG. 2E) was found to significantly correlate with decrease in Mayo score (P-value=0.005, R²=0.40; P-value=0.031, R²=0.23, respectively). Together these data (human cohort and humanized mouse model) identify a core set of transferable microbiota that are immune relevant and highlight the potential role for IgA-coated Odoribacter as a member of the CTM that associates with FMT clinical response. Specific pathogen free (SPF) Rag2^{-/-} mice were colonized with 2×10⁹ CFU of patient-derived O. splanchnicus isolate or media control following adoptive transfer of CD4+CD45RB^{high} T cells to induce transfer T cell colitis. Body weight was reported and data contained at least four mice per experimental group and error bars representing SEM. P-value were examined by repeated measures ANOVA.

IgA-coating of gut microbiota can be T cell-dependent (TD) or T cell-independent (TI). While high affinity IgA coating of certain key pathobionts are TD, gut commensal-induction of IgA can be of low affinity and TI. To evaluate the role for T cells in IgA-coating, donor material was transferred to germ-free TCR βδ^{-/-} and het control mice and IgA-seq was performed (FIG. 8A). Microbial engraftment of donor material was similarly achieved in the TCR βδ^{-/-} mice (FIG. 10A) and no significant differences in beta diversity were found between TCR βδ^{-/-} and het controls (P-value=0.21, PERMANOVA, FIG. 10B). CTM taxa enriched in IgA-coated fraction in het controls were similarly enriched in IgA-coated fraction of TCR βδ^{-/-} mice (FIGS. 2D and 10C). Collectively, these results reveal that members of the CTM induce recipient IgA responses in a TI manner.

Odoribacter splanchnicus Induces Colonic iTregs and Ameliorates T Cell Colitis

To evaluate the potential mechanistic role for Odoribacter splanchnicus in the clinical response, O. splanchnicus was isolated by cloning single cell isolates from IgA− coated recipient samples. Strains were taxonomically classified by 16SrRNA sequencing and confirmed by whole genome sequencing. The complete genome of a patient-derived 0. splanchnicus was assembled de novo into seven contigs (4,712,004 base pairs) and most closely aligned to the reference strain O. splanchnicus DSM (FIG. 11). To directly evaluate the intestinal immunity mediated by UC-derived O. splanchnicus, we colonize 6- to 8-week old wild-type (WT) germ-free mice with UC-derived O. splanchnicus. Successful colonization was assessed 7-10 days later (FIG. 12A). Analysis of colonic CD4+ T cells revealed a significant increase in RORγt+ and RORγt+ FoxP3+ cells following 20 days of colonization (FIGS. 3A and 3B; P-value=0.008 and P-value<0.0001, respectively). Consistent with the increase observed in RORyt+ CD4+ T cells, IL-17+ CD4+ T cells were also increased in colonic lamina propria of mono-colonized mice (FIGS. 3C and 3D; P-value<0.0001). No significant effect in IFNγ+ CD4+ T cells was observed (FIGS. 3C and 3D; P-value=x).

We next evaluated if the mucosal T cell response from patient-rectal biopsies correlate with the relative abundance of Odoribacter-IgA+. Although our previous report shows an overall decrease in rectal Foxp3+ CD4+ T cells post-FMT, Foxp3+ CD4+ T cells positively correlated with both Odoribacter-IgA+ relative abundance on WK4 post-FMT (P-value=0.004, R²=0.39, FIG. 12B), and also with increase in Odoribacter post FMT (Δ Odoribacter, P-value=0.007, R²=0.35, FIG. 3E). No significant correlation was found for RORyt, IL-17, IFNγ and IL-17+IFNγ+ CD4+ T cells (FIG. 12C).

Given the impact of UC-derived O. splanchnicus on mucosal T cell function at steady state, we tested the impact of UC-derived O. splanchnicus on transfer T cell colitis. Rag2^{-/-} specific-pathogen-free (SPF) mice were colonized and then challenged with sort-purified CD4+CD45RB^{high} naive T cells from WT mice. While control mice lost weight, O. splanchnicus colonization showed comparatively less weight loss (FIG. 3F, P-value=0.035).

These results suggest a mechanistic contribution of O. splanchnicus in controlling T cell inflammation in colitis.

IL-10 induced by O. splanchinicus is required to limit inflammatory Th17 cells and colitis

IL-10 is a key regulatory cytokine for intestinal homeostasis and regulatory T cell function. Mono-colonization of germ free WT mice with patient-derived O. splanchnicus was sufficient to induce Il10 expression in colonic lamina propria cells (FIG. 4A). To determine the role of IL-10 in O. splanchnicus T cell immunity, we mono-colonized IL10-deficient mice with O. splanchnicus for 18-20 days. UC-derived O. splanchnicus robustly and equivalently colonized the intestine of IL10-deficient mice (FIG. 12A). Even in IL10-deficient mice, O. splanchnicus induced both colonic iTreg and RORγt+ CD4+ Th17 cells (FIG. 4B), but the relative proportion of Th17 cells over iTreg increased significantly compared to WT mice (FIGS. 4C and 13). To assess the role of IL-10 in regulating the balance of iTreg and Th17 immunity under colitis condition, WT and IL10-deficient mice were colonized with UC-derived O. splanchnicus followed by 6 days exposure to 2% DSS. While WT GF mice succumbed to DSS-induced colitis, O. splanchnicus colonization was sufficient to rescue these mice (P value=0.045, Gehan-Breslow-Wilcoxon test, FIG. 4D); however, in contrast to WT mice, O. splanchnicus colonization failed to prevent death and weight loss in IL10-deficient mice (FIGS. 4D, E, F). Analysis of the lamina propria T cell populations of IL10-deficient mice revealed an increase in single positive RORγt and IL-17+ CD4+ T cells as well as the relative proportion of Th17 cells:iTreg (FIGS. 4G, I) compared to WT mice.

To test the role for T cells in Odoribacter-dependent protection, we colonized RAG-deficient mice. Demonstrating a role for lymphocytes in mediating this response, Odoribacter did not protect RAG-deficient mice from DSS (FIG. 4H). These results highlight a key role for O. splanchnicus-induced IL-10 in maintaining the balance of T cell activation and inflammation in colitis.

SCFA Production by O. splanchnicus Protects Against Colitis

Short-chain fatty acids (SCFAs) are bacterial metabolic products known to play a critical role in host-intestinal immunity and function. To determine the potential role for SCFA in mediating the impact of O. splanchnicus in our mouse models, germ free mice were mono-colonized with O. splanchnicus or another FMT-derived bacterium, Alistipes finegoldii, which is not part of the CTM and not a SCFA producer. As expected, a significantly higher abundance of SCFA, as measured by precursors acetyl-coA, butyrl Co-A and propionyl-coA, was detected in feces of mice mono-colonized with UC-derived O. splanchnicus compared to UC-derived A. finegoldii and germ free controls (FIG. 5A). In contrast to UC-derived O. splanchnicus, the non-SCFA producer UC-derived A. finegoldii was not able to protect mice against DSS induced colitis (FIG. 5B).

To evaluate whether this protection observed in colonization with UC-derived O. splanchnicus was mediated by SCFA, butyrate receptor (GPR109a) deficient mice and littermate controls were colonized with O. splanchnicus or media control and subjected to 2% DSS treatment (FIG. 5C). In contrast to controls, GPR109a-deficient mice colonized with O. splanchnicus lost weight and had significantly higher fecal calprotectin.

### Discussion

There is a critical need to define the composition and mechanism of transferable microbiota associated with clinical response to optimize the efficacy of microbial transfer for the treatment of UC. Our results presented here provide the first strain level analysis of FMT to define a core transferable microbiota associated with clinical response. Although diversity as a measure of engraftment have correlated with response to C diff likely through a mechanism of colonization resistance, diversity alone is not a robust predictor of clinical response in UC. Using deep metagenomic sequencing and strain tracking algorithms, we defined a core set of donor-derived strains transferred to the recipient; however, despite efficient engraftment, this overall core did not distinguish responders from non-responders. In contrast, responders engrafted a unique set of 12 donor-derived strains (shown in FIG. 1H) and the relative abundance of this correlated with the change in Mayo score. Examples include Streptococcus salivarius, Ruminococcus bromii, Roseburia intestinalis, Roseburia hominis, Odoribacter splanchnicus, Lachospiraceae bacterium 5 1 63FAA, Eubacterium biforme, Collinsella aerofaciens, Catenibacterium mitsuokai, Bilophila, Bifidobacterium adolescentis, and Bifidobacterium longum. Moreover, pre-transplant responders had lower abundance of CTM which changed significantly following transplant. These data support a potential prognostic and biomarker metric to be evaluated in future studies.

In addition to transferability, rational design of microbial therapy for UC should focus on microbes that engage the immune system and help to restore intestinal homeostasis. IgA-seq has been developed as a method to identify sentinel microbiota that interact with the mucosal immune system. Previous studies have used IgA-seq to identify IBD-associated pathobionts, but also IgA-coated commensal microbiota, the latter of which is primarily Tcell-independent. Here, we identified a core set donor-derived taxa coated with IgA in the recipient and validation in a mouse model highlights enrichment of a subset of these taxa by TI IgA-coating. Of these microbes, O. splanchnicus is this only microbe within the responder core that correlates with clinical response. IgA coating may promote durable engraftment as well as spatial location of the transferable strains in a complex microbiota.

Odoribacter has been reported to be expanded in resolving IBD but the mechanism is unclear. Our findings now reveal that this strain may work through both cellular and metabolic effectors in limiting colitis. IL-10 is key regulator pathway in IBD and O. splanchnicus induction of IL-10 promotes iTreg and restraining inflammatory Th17 cells in the lamina propria. With the efficacy of biologic therapy targeting IL-23 and inflammatory Th17 cells for UC, these findings may offer a durable non-biologic approach for this pathway of disease.

In addition to the IL-10 dependent T cell regulation, we define a key functional contribution of O. splanchnicus production of butyrate acting through gpr109. The function of butyrate can have a pleiotropic impact in promoting healing during colitis by acting direct on epithelial cells and by modulating regulatory T cell responses. Although clinical response did not correlate with fecal levels of SCFAs in our cohort, it is important to recognize that production and consumption of these are likely achieved locally. As such the binding of IgA may promote this local concentration of SCFA at the epithelial cell surfaces. Moreover, the metabolic function of the transferable bacteria raises the possibility that dietary manipulation to promote butyrate may enhance engraftment and clinical efficacy.

Collectively, this work provides the first evidence of a donor-derived strains that correlate with clinical response to FMT in UC. IgA-seq highlights the role for T cell independent IgA-coating of O. splanchnicus from within this core which mechanistically promotes protection through both cellular and metabolic function. This work identifies mechanistic features of a rational designed therapeutic strategy for UC and strategies to enhance therapeutic efficacy of this desperately needed therapeutic modality.

While the present invention has been described through various specific embodiments, routine modification to these embodiments will be apparent to those skilled in the art, which modifications are intended to be included within the scope of this disclosure.

1. A method for treatment of inflammatory bowel disease (IBD) comprising administering to an individual in need of treatment a composition comprising Odoribacter and/or Alistipes bacteria.

2. The method of claim 1, wherein the only bacteria present in the composition are Odoribacter, Alistipes or both.

3. The method of claim 1, wherein the bacteria are capable of inducing fecal IgA reactivity.

4. The method of claim 1, wherein the bacteria is Odoribacter splanchnicus.

5. The method of claim 1, wherein the bacteria is Alistipes shahii.

6. The method of claim 1, wherein the composition further comprises one or more of Streptococcus salivarius, Ruminococcus bromii, Roseburia intestinalis, Roseburia hominis, Odoribacter splanchnicus, Lachospiraceae bacterium 51 63FAA, Eubacterium biforme, Collinsella aerofaciens, Catenibacterium mitsuokai, Bilophila, Bifidobacterium adolescentis, and Bifidobacterium longum.

7. The method of claim 1, further comprising a fiber component.

8. The method of claim 1, wherein the composition is administered orally or as an enema or as an infusion into the upper gastrointestinal tract.

9. The method of claim 1, wherein the total amount of bacteria is from 1 billion to 100 billion per dose.

10. The method of claim 7, wherein the fiber is soluble or insoluble fiber.

11. The method of claim 1, wherein the composition is freeze-dried.

12. The method of claim 1, wherein the IBD is ulcerative colitis.

13. The method of claim 1, wherein the individual has a Mayo score of from 4 to 10.

14. A freeze-dried composition comprising or consisting essentially of bacteria Odoribacter and/or Alistipes.

15. The composition of claim 14, wherein the only bacteria present in the composition are Odoribacter, Alistipes or both.

16. The composition of claim 14, wherein the Odoribacter bacteria are Odoribacter splanchnicus.

17. The composition of claim 14, wherein the Alistipes bacteria are Alistipes shahii.

18. The composition of claim 14, further comprising one or more of Streptococcus salivarius, Ruminococcus bromii, Roseburia intestinalis, Roseburia hominis, Odoribacter splanchnicus, Lachospiraceae bacterium 5 1 63FAA, Eubacterium biforme, Collinsella aerofaciens, Catenibacterium mitsuokai, Bilophila, Bifidobacterium adolescentis, and Bifidobacterium longum.

19. A pharmaceutical composition comprising bacteria Odoribacter and/or Alistipes.

20. The pharmaceutical composition of claim 19, wherein the Odoribacter bacteria are Odoribacter splanchnicus, and the Alistipes bacteria are Alistipes shahii, and wherein the pharmaceutical composition optionally further comprises one or more of Streptococcus salivarius, Ruminococcus bromii, Roseburia intestinalis, Roseburia hominis, Odoribacter splanchnicus, Lachospiraceae bacterium 5 1 63FAA, Eubacterium biforme, Collinsella aerofaciens, Catenibacterium mitsuokai, Bilophila, Bifidobacterium adolescentis, and Bifidobacterium longum.
